(19) United States
(12) Patent Application Publication
(10) Pub. No.: US 2024/0209461 A1
(43) Pub. Date: Jun. 27, 2024

(54) COMPOSITIONS AND METHODS FOR STORING A BIOLOGICAL SAMPLE

(71) Applicant: GRIFOLS DIAGNOSTIC SOLUTIONS INC., Emeryville, CA (US)

(72) Inventors: Jeffrey M. LINNEN, San Diego, CA (US); Vanessa BRES, San Diego, CA (US); Lansha PENG, San Diego, CA (US); Lourdes PALACIOS MARTIN, Derio (ES)

(21) Appl. No.: 18/555,706

(22) PCT Filed: Apr. 12, 2022
(86) PCT No.: PCT/IB2022/053406

§ 371 Date: Oct. 16, 2023

Related U.S. Application Data
(60) Provisional application No. 63/175,961, filed on Apr. 16, 2021.

(51) Int. Cl. C12Q 1/70; C12Q 1/6806; G01N 1/10

(52) U.S. Cl. CPC C12Q 1/701; C12Q 1/6806; G01N 1/10; G01N 2001/1056

(57) ABSTRACT

Aqueous compositions are for storing a biological sample for subsequent nucleic acid testing. The compositions include a first component, which includes an anionic detergent in an amount from about 1% to 20% (w/v), a Group I metal hydroxide in an amount from about 1% to 5% (w/v), a chelating agent in an amount from about 0.5% to 5% (w/v), and a first buffer. The composition further includes a diluting component selected from water, a second buffer, a Transport Medium, Sodium Chloride (aq), and combinations. The first component can be diluted up to a maximum of about 50% (v/v) with the diluting component.

### PRIORITY AND CROSS REFERENCE TO RELATED APPLICATIONS

This application is the U.S. National Phase Application under 35 U.S.C. § 371 of International Application No. PCT/IB2022/053406, file on Apr. 12, 2022, and published as WO2022/219514A1 on Oct. 20, 2022, which claims the benefit of U.S. Application No. 63/175,961, filed Apr. 16, 2022. Any and all applications for which a foreign or a domestic priority is claimed is/are identified in the Application Data Sheet filed herewith and is/are hereby incorporated by reference in their entireties under 37 C.F.R. § 1.57.

### TECHNICAL FIELD

The present invention relates to aqueous compositions for storing a biological sample for subsequent nucleic acid testing comprising an anionic detergent, a chelating agent, a group I metal hydroxide and a buffer. The composition of the present invention provides for a) collection of samples, b) lysis of viruses, cells or tissues to free the nucleic acids from cellular debris and extraneous biomolecules, which results in c) inactivation of viruses, bacteria, fungi, parasites and other microbes present in that sample, d) protection of the nucleic acids from degradation by nuclease activity, and e) preservation of the nucleic acids for subsequent isolation, detection, amplification, and/or molecular analysis. Said five functions can be achieved by using a single composition and in a single reaction vessel, and the resultant sample may be stored at room temperature for extended periods without significant degradation of the polynucleotides contained within the sample.

### BACKGROUND

The current COVID-19 pandemic has brought out the urgent need for rapid and secure protocols for diagnosis of viral pathogens in a large number of individuals. COVID-19 is caused by SARS-CoV-2, a positive-sense single-stranded RNA virus. Diagnosis of this and other common diseases caused by virus and other pathogens are based on analysis of the pathogen's nucleic acids. The development of nucleic-acid-based detection platforms, such as real-time PCR, transcription-mediated amplification (TMA), ligase chain reaction (LCR), microarrays, Next Generation Sequencing (NSG), and pathogen gene chips, has drastically changed the field of clinical molecular diagnostics. However, nucleic acids in a biological sample quickly degrade and/or denature at room temperature and when diagnostic analysis are to be conducted, the ability to keep said nucleic acids stable often determines whether the nucleic acids can be successfully analyzed. This is even more important, when the desired nucleic acids for downstream analysis include ribonucleic acid (RNA), which is particularly susceptible to degradation, e.g., by endonuclease and exonuclease activity.

Another significant concern when working with biological specimens is the potential inoculation, release, or dissemination of live infectious pathogens or biological agents from the specimen into the environment. If the sample is kept viable and/or biologically intact to preserve its integrity for testing, individuals involved in the collection, transfer, and testing process are potentially exposed to highly dangerous contagions. As a result, the required safety measures typically increase the expense and effort required to move such samples from one location to another and to complete their analysis.

Accordingly, there is a need for safe collection, transport and storage compositions that preserve the integrity of the nucleic acids of even a dangerous biological specimen, typically for further molecular analysis or diagnostic testing, without posing a risk to workers.

Current protocols for handling biological samples containing nucleic acids from individuals suspected from a viral or bacterial infection includes the use of a collecting medium which allows collection of the sample and preservation of the nucleic acids until diagnostic or molecular biology tests are performed.

For detection of SARS-COV-2 and other respiratory viruses, collection of biological samples is usually carried out by using a swab that is then introduced in a tube containing the collecting medium, often called Viral Transport Medium (VTM). VTM typically contains buffered proteins (serum, albumin or gelatin) and antibiotics to suppress the growth of contaminating bacteria and fungi. For example, the CDC recommended viral transport medium (VTM) for SARS-COV-2 (Centers for Disease Control and Prevention SOP #: DSR-052-05) containing Hanks Balanced Salt Solution (HBSS), fetal bovine serum (FBS), gentamicin and Amphotericin B.

Commercially prepared VTM are available in screw cap plastic tubes. Nasal, nasopharyngeal or oropharyngeal swabs are introduced directly into the tube containing the VTM solution until the molecular test is to be performed. Then, the tube is vortexed and the sample is transferred to a secondary tube containing an extraction buffer. Said extraction buffer generally contains the necessary reagents to inactivate the virus, lyse the cells to free the nucleic acids from cellular debris and other biomolecules and protect the nucleic acid from degradation by endonuclease activity. Said transfer must be carried out under safety cabinet to avoid spreading the virus, which increases the steps before the molecular test and slows down the preparation of samples. Thus, the currently available protocols for collecting and treating samples have been proved insufficient to meet the needs of a pandemic scenario, but also other situations where a high volume of samples have to be quickly collected and processed.

On top of that, the extraction buffer often contains harmful chaotropic agents, such as guanidine thiocyanate, which acts as a denaturant agent for macromolecules, inactivating the viruses. Guanidine thiocyanate is able to decrease the degradation of RNA molecules in samples, by inactivating RNAses, therefore preserving the integrity of the nucleic acid, which is fundamental for application in molecular diagnostic methodologies in which RNA integrity is essential. However, said chaotropic agents are harmful at contact with the skin and can further interact with other reagents frequently used in the testing devices, such as sodium hypochlorite, producing toxic gases.

Thus, when it is necessary to rapidly collect, preserve and analyze a large number of samples suspected of containing a pathogen, it is very important to reduce the number of steps of the protocol while reducing the risk of spreading the pathogen.

The inventors of the present invention have developed a new composition that surprisingly inactivates any pathogen present in a sample while preserving the nucleic acid for further molecular test. In addition, said composition can be used directly to collect the sample without requiring any transfer to a second composition or buffer, as the composition itself is also able to lyse the viruses and cells and to inactivate the endonucleases and exonucleases for preserving nucleic acids. In addition, said composition is compatible with nucleic-acid-based detection platforms, in particular with commonly used sterilization agents such as sodium hypochlorite, and reduces health risk associated with sample manipulation.

The present invention also encompasses methods employing said compositions that may advantageously improve conventional collection, lysis, inactivation, storage and preservation methods for the preparation of nucleic acids from one or more biological sources.

### SUMMARY

The present invention refers to aqueous compositions for storing a biological sample for subsequent nucleic acid testing, the composition comprising a first component comprising: i) an anionic detergent in an amount from about 1% to 20% (w/v), ii) a Group I metal hydroxide in an amount from about 1% to 5% (w/v), iii) a chelating agent in an amount from about 0.5% to 5% (w/v), and iv) a first buffer, wherein the composition further comprises a diluting component selected from the group consisting of water, a second buffer, a Transport Medium, a Sodium Chloride (aq), and combinations thereof, wherein the first component is diluted up to a maximum of about 50% (v/v) with the diluting component.

In some embodiments, the present invention refers to an aqueous composition for storing a biological sample for subsequent nucleic acid testing, the composition comprising: an anionic detergent in an amount from about 1% to 20% (w/v), a Group I metal hydroxide in an amount from about 1% to 5% (w/v), a chelating agent in an amount from about 0.5% to 5% (w/v), and a first buffer, wherein the composition is diluted to a maximum of about 50% (v/v) with a diluting component selected from the group consisting of water, a second buffer, a Transport Medium and Sodium Chloride (aq), or any combination thereof.

In some embodiments, the diluting component is Sodium Chloride (aq). In some preferred embodiments, the Sodium Chloride (aq) is 0.9% (w/v) Sodium Chloride.

In some embodiments, anionic detergent comprises a C₅-C₂₀ alkyl sulfate anion, a C₅-C₂₀ alkenyl sulfate anion, a C₅-C₂₀ alkynyl sulfate anion, or any combination thereof. In some preferred embodiments, the detergent comprises a lauryl sulfate anion.

In some embodiments, the first buffer is selected from Tris, MES, Bis-Tris, HEPES, MOPS, citrate, sodium bicarbonate, sodium phosphate, and combinations thereof. In some preferred embodiments, the first buffer comprises HEPES. In some embodiments, the first buffer is present in an amount from about 5% to 30% (w/v). In some embodiments, the buffer is present in a sufficient amount to maintain a pH between 5 and 10.

In some embodiments, the Group I metal hydroxide is selected from lithium hydroxide, sodium hydroxide, potassium hydroxide, and combinations thereof. In some embodiments, the Group I metal hydroxide comprises lithium hydroxide.

In some embodiments, the chelating agent is selected from EGTA, HEDTA, DTPA, NTA, EDTA, succinic acid, citrate anhydrous, sodium citrate, calcium citrate, ammonium citrate, ammonium bicitrate, citric acid, diammonium citrate, potassium citrate, magnesium citrate, ferric ammonium citrate, lithium citrate, or combinations thereof. In some embodiments, the chelating agent is selected from succinic acid, EGTA, EDTA, and combinations thereof.

In some embodiments of the aqueous composition of the present invention, the first buffer is present in an amount from about 10% to 25% (w/v), the detergent is present in an amount from about 2% to 15% (w/v), the chelating is present in an amount from about 1% to 4% (w/v), and the hydroxide is present in an amount from about 1% to 4% (w/v).

In some embodiments, the pH of the aqueous composition is between 5 and 10. In some embodiments, the pH of the aqueous composition is between 6 and 9.

In some embodiments, the aqueous composition of the present invention further comprises an antifoaming agent in amount from about 50 μl/L to 750 μl/L. In some preferred embodiments, said antifoaming agent comprises a silicone polymer, a polysorbate, an organic polyether dispersion or combinations thereof.

In some embodiments, the aqueous composition of the present invention further comprises a population of polynucleotides that comprises RNA, DNA, or any combination thereof.

In some embodiments, the RNA comprises an in vitro synthetized transcript.

In another aspect, the present invention refers to an aqueous composition for storing a biological sample for subsequent nucleic acid testing, the composition comprising: i) an anionic detergent in an amount from about 1% to 20% (w/v), ii) a Group I metal hydroxide in an amount from about 1% to 5% (w/v), iii) a chelating agent in an amount from about 0.5% to 5% (w/v), and iv) a first buffer, wherein the composition comprises an antifoaming agent in amount from about 50 μl/L to 750 μl/L.

In some embodiments, the antifoaming agent comprises a silicone polymer, a polysorbate, an organic polyether dispersion, or any combination thereof.

In some embodiments, the anionic detergent comprises a C5-C20 alkyl sulfate anion, a C5-C20 alkenyl sulfate anion, a C5-C20 alkynyl sulfate anion, or any combination thereof. In some preferred embodiments the detergent comprises a lauryl sulfate anion.

In some embodiments, the first buffer is selected from Tris, MES, Bis-Tris, HEPES, MOPS, citrate, sodium bicarbonate, sodium phosphate, and combinations thereof. In some preferred embodiments, the first buffer comprises HEPES.

In some embodiments, the first buffer is present in an amount from about 5% to 30% (w/v).

In some embodiments, the first buffer is present in a sufficient amount to maintain a pH between 5 and 10.

In some embodiments, the Group I metal hydroxide is selected from lithium hydroxide, sodium hydroxide, potassium hydroxide, and combinations thereof. In some preferred embodiments, the Group I metal hydroxide comprises lithium hydroxide.

In some embodiments, the chelating agent is selected from EGTA, HEDTA, DTPA, NTA, EDTA, succinic acid, citrate anhydrous, sodium citrate, calcium citrate, ammonium citrate, ammonium bicitrate, citric acid, diammonium citrate, potassium citrate, magnesium citrate, ferric ammonium citrate, lithium citrate, and combinations thereof. In some preferred embodiments, the chelating agent is selected from succinic acid, EGTA, EDTA, and combinations thereof.

In some embodiments, the first buffer is present in an amount from about 10% to 25% (w/v), the anionic detergent is present in an amount from about 2% to 15% (w/v), the chelating is present in an amount from about 1% to 4% (w/v), and the Group I metal hydroxide is present in an amount from about 1% to 4% (w/v).

In some embodiments, the pH of the aqueous composition is between 5 and 10. In some preferred embodiments, the pH of the aqueous composition is between 6 and 9.

In some embodiments, the composition is diluted up to a maximum of about 50% (v/v) with a component selected from the group consisting of water, a second buffer, a Transport Medium, Sodium Chloride (aq), and combinations thereof. In some preferred embodiments, the composition is diluted with Sodium Chloride (aq).

In some embodiments, the composition of the present invention further comprises a population of polynucleotides that comprises RNA, DNA, or any combination thereof. In some preferred embodiments the RNA comprises an in vitro synthetized transcript.

The present invention also relates to a method for obtaining a population of polynucleotides from a sample suspected of containing nucleic acids, said method comprising contacting the sample with the aqueous composition of the present invention at a temperature ranging from 2° C. to 40° C.

In some embodiments, the sample is a biological sample or an environmental sample. In some embodiments, the nucleic acids are RNA and/or DNA. In some preferred embodiments, the RNA comprises an in vitro synthetized transcript. In some embodiments, the nucleic acids are from at least one pathogen. In some preferred embodiments, at least one pathogen is a fungi, a bacteria, a parasite or a virus. In preferred embodiments, the at least one pathogen is a fungi. In preferred embodiments, the at least one pathogen is a bacteria. In some preferred embodiments, the at least one pathogen is a parasite. In preferred embodiments, the at least one pathogen is a virus. In preferred embodiments, the virus is SARS-COV-2.

In some embodiments, the method for obtaining a population of polynucleotides from a sample suspected of containing nucleic acids further comprises an amplification reaction to amplify at least a target nucleic acid and the detection of at least one target of the resulting amplification product.

The present invention also relates to a method for preserving the integrity of a population of polynucleotides in a sample, comprising contacting the sample with the aqueous compositions according to the present invention, at a temperature ranging from 4° C. to 40° C. In some preferred embodiments, the sample, after being contacted with the aqueous compositions of the present invention, is stored at a temperature ranging from −25° C. to 40° C. to for at least 1 hour up to 6 months, up to 9 months, up to 12 months, or up to 26 months. In some preferred embodiments, the sample, after being contacted with the aqueous compositions, is stored at −20° C., 4° C., RT or 30° C.

In some embodiments, the sample is a biological sample or an environmental sample. In some embodiments, the population of polynucleotides is a population of RNA and/or DNA. In some preferred embodiments, the RNA comprises an in vitro synthetized transcript. In some embodiments, the population of polynucleotides are from at least one pathogen. In some preferred embodiments, at least one pathogen is a fungi, a bacteria, a parasite or a virus. In preferred embodiments, the at least one pathogen is a fungi. In preferred embodiments, the at least one pathogen is a bacteria. In some preferred embodiments, the at least one pathogen is a parasite. In preferred embodiments, the at least one pathogen is a virus. In preferred embodiments, the virus is SARS-COV-2.

The present invention also relates to a method for inactivating a sample comprising at least one pathogen, the method comprising contacting the sample with the aqueous compositions according to the present invention, wherein the aqueous compositions represents at least 30% (v/v) of the resulting mixture. In some embodiments, the sample is contacted with the aqueous compositions at 4° C., RT or 30° C. In some embodiments, the sample is a biological sample or an environmental sample. In some embodiments, the at least one pathogen is a fungi, a bacteria, a parasite or a virus. In preferred embodiments, the at least one pathogen is a fungi. In preferred embodiments, the at least one pathogen is a bacteria. In some preferred embodiments, the at least one pathogen is a parasite. In preferred embodiments, the at least one pathogen is a virus. In preferred embodiments, the virus is SARS-COV-2.

The present invention also relates to a method for collecting a sample comprising contacting the sample with the aqueous compositions according to the present invention, wherein the aqueous compositions represents at least 30% (v/v) of the resulting mixture. In some embodiments, the sample is a biological sample or an environmental sample.

In some embodiments, the sample is directly introduced into a collection device or a collection vessel comprising the aqueous compositions according to the present invention.

In other embodiments, the sample is collected using a swab and introducing it into a collection device or a collection vessel comprising the aqueous compositions according to the present invention. In some preferred embodiments the swab is discarded after rubbing it against the collection device or the collection vessel.

In some embodiments, the sample comprises at least one pathogen. In some embodiments, the at least one pathogen is a fungi, a bacteria, a parasite or a virus. In preferred embodiments, the at least one pathogen is a fungi. In preferred embodiments, the at least one pathogen is a bacteria.

In some preferred embodiments, the at least one pathogen is a parasite. In preferred embodiments, the at least one pathogen is a virus. In preferred embodiments, the virus is SARS-CoV-2.

The present invention also relates to a collection device or vessel comprising the aqueous compositions according to the present invention.

The present invention also relates to a sample collection kit comprising a collection device or a collection vessel and the aqueous compositions according to the present invention. In some embodiments, said sample collection kit further comprises a swab, a curette, or a culture loop.

The present invention also relates to the use of the aqueous compositions according to the present invention to collect a sample suspected of containing a target nucleic acid. In some embodiments, the sample is a biological sample or an environmental sample. In some embodiments, the sample comprises at least one pathogen. In some embodiments, the at least one pathogen is a fungi, a bacteria, a parasite or a virus. In preferred embodiments, the at least one pathogen is a fungi. In preferred embodiments, the at least one pathogen is a bacteria. In some preferred embodiments, the at least one pathogen is a parasite. In preferred embodiments, the at least one pathogen is a virus. In preferred embodiments, the virus is SARS-COV-2.

### BRIEF DESCRIPTION OF THE DRAWINGS

FIG. 1 shows a picture of a 96-well plate with SARS-COV-2 infected and non-infected Vero cells stained with crystal violet.

FIGS. 2 to 5 show stability of positive SeraCare and BEI samples stored at 4° C. or 30° C. up to 6 months. FIG. 2 shows % of reactivity; FIG. 3 shows Analyte RLU values versus time; FIG. 4 shows Internal Control (IC) RLU values versus time, and FIG. 5 shows Analyte-S/CO RLU values versus time.

FIG. 6 shows stability of Babesia IVT samples (500 c/mL) stored at 5° C. up to 12 months.

FIG. 7 shows stability of Babesia IVT samples (100, 30 and 500 c/mL) stored at −20° ° C. up to 26 months.

### DETAILED DESCRIPTION

The words “comprises/comprising” and the words “having/including” when used herein with reference to the present invention are used to specify the presence of stated features, integers, steps or components but do not preclude the presence or addition of one or more other features, integers, steps, components or groups thereof.

It should be appreciated by those skilled in the art that the specific embodiments disclosed herein should not be read in isolation, and that the present specification intends for the disclosed embodiments to be read in combination with one another as opposed to individually. As such, each embodiment may serve as a basis for modifying or limiting other embodiments disclosed herein.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “10 to 100” should be interpreted to include not only the explicitly recited values of 10 to 100, but also include individual value and sub-ranges within the indicated range. Thus, included in this numerical range are individual values such as 10, 11, 12, 13 . . . 97, 98, 99, 100 and sub-ranges such as from 10 to 40, from 25 to 40 and 50 to 60, etc. This same principle applies to ranges reciting only one numerical value, such as “at least 10”. Furthermore, such an interpretation should apply regardless of the breadth of the range or the characteristics being described.

### Compositions of the Present Invention

In a first aspect, the present invention refers to an aqueous composition for storing a biological sample for subsequent nucleic acid testing, the composition comprising a first component comprising:
i) an anionic detergent in an amount from about 1% to 20% (w/v), ii) a Group I metal hydroxide in an amount from about 1% to 5% (w/v), iii) a chelating agent in an amount from about 0.5% to 5% (w/v), and iv) a first buffer, wherein the composition further comprises a diluting component selected from the group consisting of water, a second buffer, a Transport Medium, Sodium Chloride (aq), and combinations thereof, wherein the first component is diluted up to a maximum of about 50% (v/v) with the diluting component.

In a second aspect, the present invention refers to aqueous compositions for storing a biological sample for subsequent nucleic acid testing, the composition comprising: i) an anionic detergent in an amount from about 1% to 20% (w/v), ii) a Group I metal hydroxide in an amount from about 1% to 5% (w/v), iii) a chelating agent in an amount from about 0.5% to 5% (w/v), and iv) a first buffer, wherein the composition comprises an antifoaming agent in amount from about 50 μl/L to 750 μl/L.

In another aspect, the present invention refers to an aqueous composition for storing a biological sample for subsequent nucleic acid testing, the composition comprising: an anionic detergent in an amount from about 1% to 20% (w/v), a Group I metal hydroxide in an amount from about 1% to 5% (w/v), a chelating agent in an amount from about 0.5% to 5% (w/v), and a first buffer, wherein the composition is diluted to a maximum of about 50% (v/v) with a diluting component selected from the group consisting of water, a second buffer, a Transport Medium and Sodium Chloride (aq), or any combination thereof.

In some preferred embodiments, the Sodium Chloride (aq) is 0.9% (w/v) Sodium Chloride.

In some embodiments, the anionic detergent comprises a C₅-C₂₀ alkyl sulfate anion, a C₅-C₂₀ alkenyl sulfate anion, a C₅-C₂₀ alkynyl sulfate anion, or combinations thereof. In some embodiments the detergent comprises a lauryl sulfate anion. In some embodiments, the detergent comprises lithium lauryl sulfate (LLS) or sodium dodecyl sulfate (SDS). In some embodiments, the detergent comprises LLS.

In some embodiments, the amount of anionic detergent in the aqueous compositions of the present invention is from about 1% to 20% (w/v), from about 2% to 20% (w/v), from about 3% to 20% (w/v), from about 5% to 20% (w/v), from about 7% to 20% (w/v), from about 9% to 20% (w/v), from about 10% to 20% (w/v), from about 15% to 20% (w/v), from about 1% to 15% (w/v), from about 1% to 12% (w/v), from about 1% to 10% (w/v), from about 1% to 9% (w/v), from about 1% to 7% (w/v) or from about 1% to 5% (w/v). In some embodiments, the amount of anionic detergent in the composition of the present invention is from about 2% to 15% (w/v), from about 3% to 12% (w/v), from about 5% to 10% (w/v) or from about 7% to 9% (w/v).

The term “buffer”, as used herein, refers to a weak acid or weak base used to maintain the pH of a solution. In some embodiments, the buffer is selected from tris(hydroxymethyl)aminomethane (Tris), 2-(N-morpholino)ethanesulfonic acid (MES), 1,3-bis(tris(hydroxymethyl)methylamino)propane (Bis-Tris), 4-(2-hydroxyethyl)-1-piperazineethanesulfonic acid (HEPES), 3-(N-morpholino)propanesulfonic acid (MOPS), citrate, sodium bicarbonate, sodium phosphate, and combinations thereof. In a preferred embodiment, the buffer comprises HEPES.

In some embodiments, the amount of buffer in the aqueous compositions of the present invention is from 5% to 30% (w/v), from 5% to 25% (w/v), from 5% to 20% (w/v), from 5% to 15% (w/v), from about 5% to 10% (w/v), from about 10% to 30% (w/v), from about 15% to 30% (w/v) or from about 20% to 30% (w/v). In some embodiments, the amount of buffer in the composition of the present invention is from about 10% to 25% (w/v), from about 10% to 22% (w/v), from about 12% to 20% (w/v) or from about 15% to 18% (w/v).

In some embodiments, the buffer is present in the aqueous compositions of the present invention in a sufficient amount to maintain a pH between 5 and 10. More preferably, the pH is between 6 and 9, between 6.5 and 8.5, between 7 and 8, between 7.2 and 7.8. In other embodiments, the pH is between 6 and 10, 7 and 10 or 7.5 and 10. In other embodiments, the pH is between 5 and 9, between 5 and 8, between 5 and 7.8, between 5 and 7.5, or between 5 and 7.2.

In some embodiments, the Group I metal hydroxide is selected from lithium hydroxide, sodium hydroxide, potassium hydroxide and combinations thereof. In a more preferred embodiment, the Group I metal hydroxide is lithium hydroxide.

In some embodiments, the amount Group I metal hydroxide in the composition of the present invention is from about 1% to 5% (w/v), from about 1% to 4.5% (w/v), from about 1% to 4% (w/v), from about 1% to 3.5% (w/v), from about 1% to 3% (w/v), from about 1% to 2.5% (w/v), from about 1.5% to 5% (w/v), from about 2% to 5% (w/v), from about 2.5% to 5% (w/v), from about 3% to 5% (w/v) or from about 3.5% to 5% (w/v). In some embodiments, the amount of base in the composition of the present invention is from about 1.5% to 4.5% (w/v), from about 2% to 4% (w/v), from about 2.5% to 3.5% (w/v) or from about 2.5% to 3% (w/v).

The term “chelating agent”, as used herein, refers to chemical compounds that react with metal ions to form complex ring-like structures called chelates. In some embodiments, the chelating agent is selected from ethylene glycol tetraacetic acid (EGTA), hydroxyethylethylenediaminetriacetic acid (HEDTA), diethylene triamine pentaacetic acid (DTPA), N, N-bis(carboxymethyl)glycine (NTA), ethylenediaminetetraacetic (EDTA), succinic acid, citrate anhydrous, sodium citrate, calcium citrate, ammonium citrate, ammonium bicitrate, citric acid, diammonium citrate, potassium citrate, magnesium citrate, ferric ammonium citrate, lithium citrate, and combinations thereof. In preferred embodiments, the chelating agent is selected from succinic acid, EGTA, EDTA and combination thereof. In some preferred embodiments, the chelating agent is succinic acid.

In some embodiments, the amount of chelating agent in the composition of the present invention is from about 0.5% to 5% (w/v), from about 0.5% to 4.5% (w/v), from about 0.5% to 4% (w/v), from about 0.5% to 3.5% (w/v), from about 0.5% to 3% (w/v), from about 0.5% to 2.5% (w/v), from about 0.5% to 2% (w/v), from about 0.5% to 1.5% (w/v), from about 1% to 5% (w/v), from about 1.5% to 5% (w/v), from about 2% to 5% (w/v), from about 2.5% to 5% (w/v), from about 3% to 5% (w/v), from about 3.5% to 5% (w/v) or from about 4% to 5% (w/v). In some embodiments, the amount of chelating agent in the composition of the present invention is from about 1% to 4% (w/v), from about 1.5% to 4% (w/v), from about 2% to 3% (w/v) or from about 2.5% to 3% (w/v).

In some embodiments of the aqueous compositions of the present invention the buffer is present in an amount from about 10% to 25% (w/v), the anionic detergent is present in an amount from about 2% to 15% (w/v), the chelating is present in an amount from about 1% to 4% (w/v), and the Group I metal hydroxide is present in an amount from about 1% to 4% (w/v).

In some embodiments of the aqueous compositions of the present invention the buffer is present in an amount from about 15% to 20% (w/v), the anionic detergent is present in an amount from about 5% to 10% (w/v), the chelating is present in an amount from about 2% to 3% (w/v), and the Group I metal hydroxide is present in an amount from about 2% to 3% (w/v).

In some embodiments, the aqueous compositions of the present invention have a pH between 5 and 10. More preferably, the pH of the aqueous compositions of the present invention is between 6 and 9, between 6.5 and 8.5, between 7 and 8, between 7.2 and 7.8. In other embodiments, the pH is between 6 and 10, 7 and 10 or 7.5 and 10. In other embodiments, the pH is between 5 and 9, between 5 and 8, between 5 and 7.8, between 5 and 7.5, or between 5 and 7.2.

The term “antifoaming agent” or “defoaming agent” as used herein, refers to chemical additives that reduces and hinders the formation of foam in liquid compositions. In the context of the present invention, said antifoaming agents prevent the formation of bubbles that typically result from the presence of detergents in the formulation and/or facilitate pipetting and handling of the disclosed compositions. In some embodiments, the composition of the present invention further comprises an antifoaming agent. In some embodiments, the amount of the antifoaming agent is from 50 μl/L to 750 μl/L, from 50 μl/L to 600 μl/L, from 50 μl/L to 500 μl/L, from 50 μl/L to 400 μl/L, from 50 μl/L to 300 μl/L, from 50 μl/L to 250 μl/L, from 50 μl/L to 200 μl/L, from 50 μl/L to 150 μl/L, from 75 μl/L to 750 μl/L, from 100 μl/L to 750 μl/L, from 150 μl/L to 750 μl/L, from 200 μl/L to 750 μl/L, from 250 μl/L to 750 μl/L, from 300 μl/L to 750 μl/L or from 500 μl/L to 750 μl/L. In some preferred embodiments, the amount of the antifoaming agent is from 75 μl/L to 600 μl/L, from 100 μl/L to 500 μl/L, from 150 μl/L to 400 μl/L, or from 200 μl/L to 300 μl/L. Exemplary antifoaming agent agents for the present invention include, without limitation, cocoamidopropyl hydroxysultaine, alkylaminopropionic acids, imidazoline carboxylates, betaines, sulfobetaines, sultaines, alkylphenol ethoxylates, alcohol ethoxylates, polyoxyethylenated polyoxypropylene glycols, polyoxyethylenated mercaptans, long-chain carboxylic acid esters, alkonolamides, tertiary acetylenic glycols, polyoxyethylenated silicones, N-alkylpyrrolidones, alkylpolyglycosidases, silicone polymers, polysorbates, organic polyether dispersions, or combinations thereof. In some preferred embodiment, the antifoaming agent comprises a silicone polymer, a polysorbate, an organic polyether dispersion or combinations thereof. In a more preferred embodiment, the antifoaming agent comprises a silicone polymer. In a yet more preferred embodiment, said silicone polymer is a 3-Dimensional Siloxane. In an even more preferred embodiment, the antifoaming agent comprises Foam Ban® MS-575.

The antifoaming agent can be included in the aqueous compositions, for example, when the sample is to be analyzed in a platform susceptible to make errors if bubbles are present in the sample.

The composition of the present invention is diluted to a maximum of about 50% (v/v) with a component selected from the group consisting of water, a buffer, a Transport Medium and Sodium Chloride (aq), or any combination thereof.

In some embodiments, the aqueous compositions are diluted to a maximum of about 50% (v/v) with water. In some embodiments, the aqueous compositions is diluted to a maximum of about 30% (v/v), to a maximum of about 40% (v/v), to a maximum of about 45% (v/v), to a maximum of about 55% (v/v), to a maximum of about 60% (v/v), to a maximum of about 75% (v/v), to a maximum of about 80% (v/v) or to a maximum of about 80% (v/v).

The term “transport medium”, as used herein, refers to any solution that comprises protective proteins and antimicrobial to avoid microbial grow for transport of a biological specimen. In some embodiments, the aqueous compositions are diluted to a maximum of about 50% (v/v) with transport medium. In some embodiments, the aqueous compositions is diluted to a maximum of about 30% (v/v), to a maximum of about 40% (v/v), to a maximum of about 45% (v/v), to a maximum of about 55% (v/v), to a maximum of about 60% (v/v), to a maximum of about 75% (v/v), to a maximum of about 80% (v/v) or to a maximum of about 80% (v/v).

In some embodiments, the aqueous compositions are diluted to a maximum of about 50% (v/v) with Sodium Chloride (aq). In some preferred embodiments, the Sodium Chloride (aq) is 0.9% (w/v) sodium chloride. In some embodiments, the aqueous compositions are diluted to a maximum of about 30% (v/v), to a maximum of about 40% (v/v), to a maximum of about 45% (v/v), to a maximum of about 55% (v/v), to a maximum of about 60% (v/v), to a maximum of about 75% (v/v), to a maximum of about 80% (v/v) or to a maximum of about 80% (v/v).

In some embodiments, the aqueous compositions are diluted to a maximum of about 50% (v/v) with any combination of water, a buffer, a Transport Medium and 0.9% (w/v) Sodium Chloride. In some embodiments, the aqueous compositions are diluted to a maximum of about 30% (v/v), to a maximum of about 40% (v/v), to a maximum of about 45% (v/v), to a maximum of about 55% (v/v), to a maximum of about 60% (v/v), to a maximum of about 75% (v/v), to a maximum of about 80% (v/v) or to a maximum of about 80% (v/v).

In some embodiments, the aqueous compositions of the present invention further comprise a population of polynucleotides that comprises RNA, DNA, or a combination thereof. In some embodiments, the RNA comprises an in vitro synthetized transcript. In some embodiments, said population of polynucleotides can include primers, amplification oligomers, detection oligomers, probe protection oligomers, capture probe oligomer or any other polynucleotide required for conducting an amplification reaction to amplify a target nucleic acid and/or for detecting said target nucleic acid. In other embodiments, said population of polynucleotides is a target nucleic acid.

The term “nucleic acid” refers to a multimeric compound comprising two or more covalently bonded nucleosides or nucleoside analogs having nitrogenous heterocyclic bases, or base analogs, where the nucleosides are linked together by phosphodiester bonds or other linkages to form a polynucleotide. Nucleic acids include RNA, DNA, or chimeric DNA-RNA polymers or oligonucleotides, and analogs thereof.

By “isolated” it is meant that a sample containing a target nucleic acid is taken from its natural milieu, but the term does not connote any degree of purification.

### Methods of the Present Invention

In a further aspect the present invention relates to a method for obtaining a population of polynucleotides from a sample suspected of containing nucleic acids, said method comprising contacting the sample with the aqueous compositions according to the present invention at a temperature ranging from 2° C. to 40° C. In some embodiments, said contact is done at a temperature ranging from 2° ° C. to 40° C., more preferably at a temperature ranging from 4° C. to 35° C., more preferably from 4° C. to 30° C. In some embodiments, said contact is done at room temperature. The term “room temperature” (RT), as used herein, generally refers to a range of temperature going from 15° C. to 30° C.

In some embodiments, the sample is contacted with the aqueous compositions preferably for at least 1 min, for at least 2 min, for at least 3 min, for at least 5 min, for at least 10 min or for at least 15 min.

In some embodiments, the sample is contacted with the aqueous compositions according to the present invention, for at least 1 min at a temperature ranging from 2° C. to 40° C.

The sample may be an isolated sample. Samples include “biological samples”, “environmental samples”, and sampling devices (e.g., swabs) brought into contact with biological or environmental samples or any other sample suspected of containing a pathogen nucleic acid or components thereof.

“Biological samples” include body fluids such as urine, blood, plasma, serum, peripheral blood, red blood cells, lymph node, gastrointestinal tissue, fecal matter, cerebrospinal fluid (CSF), semen, sputum, saliva, or other body fluids or materials as well as solid tissue. Biological samples also include a cell (such as cell lines, cells isolated from tissue whether or not the isolated cells are cultured after isolation from tissue, fixed cells such as cells fixed for histological and/or immunohistochemical analysis), tissue (such as biopsy material), or fluid obtained from a mammal, including from the upper respiratory tissues (such as nasopharyngeal wash, nasopharyngeal aspirate, nasopharyngeal swab, and oropharyngeal swab), from the lower respiratory tissues (such as bronchiolar lavage, tracheal aspirate, pleural tap, sputum), and tissue from any organ such as, without limitation, lung, heart, spleen, liver, brain, kidney, and adrenal glands. Nucleic acids (e.g., DNA and RNA) isolated from a cell and/or tissue, and the like are also included. In some preferred embodiments, biological samples include anterior nasal and mid-turbinate nasal swabs, nasopharyngeal (NP) and oropharyngeal (OP) swabs, nasopharyngeal washes/aspirates or nasal aspirates, and broncho alveolar lavage (BAL) specimens. In more preferred embodiments, said specimens are obtained from individuals suspected of COVID-19.

“Environmental samples” include environmental material such as surface matter, soil, water, and industrial materials, as well as material obtained from food and dairy processing instruments, apparatus, equipment, disposable, and non-disposable items.

In some embodiments of the method for obtaining a population of polynucleotides from a sample suspected of containing nucleic acids, the sample is a biological sample. In other embodiments, the sample is an environmental sample. In some embodiments, the nucleic acids are RNA and/or DNA. In some embodiments, the RNA comprises an in vitro synthetized transcript.

The term “sample” includes any specimen that may contain, or is suspected of containing, at least one pathogen or components thereof, such as nucleic acids or fragments of a pathogen nucleic acid. In some embodiments, said at least one pathogen is a fungi, a bacteria, a parasite or a virus. In some embodiments, said at least one pathogen is a virus. In other embodiments said virus is a respiratory virus. In some embodiments, said respiratory virus is from the coronaviridae family. In more preferred embodiments, said virus is SARS-COV-2. Other non-respiratory viruses are also contemplated, such as HIV, HCV, HBV, HAV, HEV, parvovirus, West Nile Virus, Usutu, Chikungunya, Dengue, or Zika, among others. In some embodiments, the at least one pathogen is a bacteria. In some embodiments, the at least one pathogen is a fungi. In some embodiments, the at least one pathogen is a parasite. Thus, other pathogens, such as Babesia or Plasmodium, are also contemplated within the scope of the present invention.

In some embodiments, the method for obtaining a population of polynucleotides from a sample suspected of containing nucleic acids further comprises an amplification reaction to amplify at least a target nucleic acid. In some embodiments, said method further comprises detecting at least a target from the resulting amplification product. In some embodiments, the amplification reaction is an isothermal amplification reaction. In more preferred embodiments, the amplification reaction is a transcription mediated amplification.

The term “nucleic-acid-based detection” or “nucleic acid testing” as used herein, refer to assays for the detection of a target sequence. In certain embodiments, said nucleic-acid-based detection assay comprises detection of a target sequence by distinguish it among others. For example, by sequencing or by utilizing one or more oligonucleotides that specifically hybridize to the target sequence. In certain embodiments, a nucleic-acid-based detection assay is an “amplification-based assay,” i.e., an assay that utilizes one or more steps for amplifying a nucleic acid target sequence. For the sake of clarity, an amplification-based assay may include one or more steps that do not amplify a target sequence, such as, for example, steps used in non-amplification-based assay methods (e.g., a hybridization assay or a cleavage-based assay). Suitable amplification methods include, for example, replicase-mediated amplification, polymerase chain reaction (PCR), quantitative PCR (qPCT), real time PCR (rt-PCT), ligase chain reaction (LCR), strand-displacement amplification (SDA), transcription-mediated or transcription-associated amplification (TMA), nucleic acid sequence based amplification (NASBA), loop mediated isothermal amplification (LAMP) and polymerase spiral reaction (PSR). Such amplification methods are well-known in the art and are readily used in accordance with the methods of the present disclosure. Other non-target amplification techniques known by the skilled person are also within the spirit of the present invention, such as strand-displacement amplification, also called whole genome amplification (WGA). In other embodiments, a nucleic-acid-based detection assay is a “non-amplification-based assay,” i.e., an assay that does not rely on any step for amplifying a nucleic acid target sequence.

“Transcription-associated amplification,” also referred to herein as “transcription-mediated amplification” (TMA), is an isothermal amplification reaction that refers to nucleic acid amplification that uses an RNA polymerase to produce multiple RNA transcripts from a nucleic acid template. TMA generally employs an RNA polymerase, a DNA polymerase, deoxyribonucleoside triphosphates, ribonucleoside triphosphates, and a template complementary oligonucleotide that includes a promoter sequence, and optionally may include one or more other oligonucleotides.

In a further aspect the present invention relates to a method for preserving the integrity of a population of polynucleotides in a sample, comprising contacting the sample with the aqueous compositions according to the present invention at a temperature ranging from 4° C. to 40° C.

In some preferred embodiments, the sample is contacted with the aqueous compositions for at least 1 min, for at least 2 min, for at least 3 min, for at least 5 min, or for at least 10 min. In some embodiments, the sample is contacted with the aqueous compositions for at least 1 day, for at least 2 days, for at least 5 days, for at least 7 days, for at least 15 days, for at least 30 days, for at least 1 month, for at least 2 months, for at least 3 months, for at least 6 months, for at least 9 months, for at least 12 months, for at least 24 months or for at least 26 months.

In some embodiments, said contact is done at a temperature ranging from 4° C. to 40° C., more preferably at a temperature ranging from 4° C. to 35° C., more preferably from 4° C. to 30° C. In some embodiments, said contact is done at room temperature. The term “room temperature”, as used herein, generally refers to a range of temperature going from 15° C. to 30° C. In some embodiments, the sample is contacted with the aqueous compositions according to the present invention, for at least 2 min at a temperature ranging from 4° C. to 30° C.

In some embodiments of the method for preserving the integrity of a population of polynucleotides in a sample, the sample, after being contacted with the aqueous compositions, is stored at a temperature ranging from −25° C. to 40° C. for at least 1 hour up to 6 months, up to 9 months, up to 12 months, or up to 26 months. In more preferred embodiments, the sample, after being contacted with the aqueous compositions, is stored at −20° C., 4° C., RT or 30° C. for at least at least 1 hour up to 6 months, up to 12 months, up to 24 months, or up to 26 months. In some embodiments, the sample, after being contacted with the aqueous compositions, is stored at a temperature ranging from 2° C. to 10° C. for at least at least 1 hour up to 9 months. In some embodiments, the sample, after being contacted with the aqueous compositions, is stored at a temperature ranging from −25° C. to −5° C. for at least at least 1 hour up to 26 months.

In some embodiments of the method for preserving the integrity of a population of polynucleotides in a sample, the sample is a biological sample as defined herein. In some preferred embodiments, biological samples include anterior nasal and mid-turbinate nasal swabs, nasopharyngeal (NP) and oropharyngeal (OP) swabs, nasopharyngeal washes/aspirates or nasal aspirates, and broncho alveolar lavage (BAL) specimens. In more preferred embodiments, said specimens are obtained from individuals suspected of COVID-19. In other embodiments, the sample is an environmental sample as defined herein.

In some embodiments, the population of polynucleotides is a population of RNA and/or DNA. In some embodiments, the population of polynucleotides is from at least one pathogen. In some embodiments, said at least one pathogen is a fungi, a bacteria, a parasite or a virus. In some embodiments, said at least one pathogen is a virus. In other embodiments said virus is a respiratory virus. In some embodiments, said respiratory virus is from the coronaviridae family. In more preferred embodiments, said virus is SARS-COV-2. Other non-respiratory viruses are also contemplated, such as HIV, HCV, HBV, HAV, HEV, parvovirus, West Nile Virus, Usutu, Chikungunya, Dengue, or Zika, among others. In some embodiments, the at least one pathogen is a bacteria. In some embodiments, the at least one pathogen is a fungi. In some embodiments, the at least one pathogen is a parasite. Thus, other pathogens, such as Babesia or Plasmodium, are also contemplated within the scope of the present invention.

In a further aspect the present invention relates to a method for inactivating a sample comprising at least one pathogen, the method comprising contacting the sample with the aqueous compositions according to the present invention, wherein the aqueous compositions represents at least 30% (v/v) of the resulting mixture.

In some embodiments, said aqueous compositions represents at least 2% (v/v) of the resulting mixture, more preferably at least 5% (v/v) of the resulting mixture, more preferably at least 10% (v/v) of the resulting mixture, more preferably at least 20% (v/v) of the resulting mixture, more preferably at least 30% (v/v) of the resulting mixture, more preferably at least 35% (v/v) of the resulting mixture, more preferably at least 40% (v/v) of the resulting mixture, more preferably at least 50% (v/v) of the resulting mixture, more preferably at least 60% (v/v) of the resulting mixture, more preferably at least 70% (v/v) of the resulting mixture, more preferably at least 80% (v/v) of the resulting mixture, more preferably at least 85% (v/v) of the resulting mixture, more preferably at least 90% (v/v) of the resulting mixture, more preferably at least 95% (v/v) of the resulting mixture, more preferably at least 98% (v/v) of the resulting mixture, more preferably at least 99% (v/v) of the resulting mixture.

In some embodiments, the sample is contacted with the aqueous compositions for at least 1 min, for at least 2 min, for at least 3 min, at least 5 min, for at least 10 min, for at least 15 min, or for at least 30 min. In some embodiments, said contact is done at a temperature ranging from 2° C. to 40° C., more preferably at a temperature ranging from 4° C. to 35° C., more preferably from 4° C. to 30° C. In some embodiments, said contact is done at room temperature. The term “room temperature”, as used herein, generally refers to a range of temperature going from 15° C. to 30° C. In some preferred embodiments, the sample is contacted with the aqueous compositions at 4° C., RT or 30° C.

In some embodiments, the sample is contacted with the aqueous compositions according to the present invention, for at least 1 min at a temperature ranging from 4° C. to 30° C.

In some embodiments of the method for inactivating a sample comprising one or more pathogens of the present invention, the sample is as defined herein. In some preferred embodiments, the sample is a biological sample as defined herein. In some preferred embodiments, biological samples include anterior nasal and mid-turbinate nasal swabs, nasopharyngeal (NP) and oropharyngeal (OP) swabs, nasopharyngeal washes/aspirates or nasal aspirates, and broncho alveolar lavage (BAL) specimens. In more preferred embodiments, said specimens are obtained from individuals suspected of COVID-19. In other embodiments, the sample is an environmental sample as defined herein.

In some embodiments, said at least one pathogen is a fungi, a bacteria, a parasite or a virus. In some embodiments, said at least one pathogen is a virus. In other embodiments said virus is a respiratory virus. In some embodiments, said respiratory virus is from the coronaviridae family. In more preferred embodiments, said virus is SARS-COV-2. Other non-respiratory viruses are also contemplated, such as HIV, HCV, HBV, HAV, HEV, parvovirus, West Nile Virus, Usutu, Chikungunya, Dengue, or Zika, among others. In some embodiments, the at least one pathogen is a bacteria. In some embodiments, the at least one pathogen is a fungi. In some embodiments, the at least one pathogen is a parasite. Thus, other pathogens, such as Babesia or Plasmodium, are also contemplated within the scope of the present invention.

In a further aspect the present invention relates to a method for collecting a sample comprising contacting the sample with the aqueous compositions according to the present invention, wherein the aqueous compositions represents at least 30% (v/v) of the resulting mixture. In some embodiments, the aqueous compositions represent at least 30% (v/v) of the resulting mixture from contacting the sample with the aqueous compositions. In some embodiments, said aqueous compositions represents at least 2% (v/v) of the resulting mixture, or at least 5% (v/v) of the resulting mixture, or at least 10% (v/v) of the resulting mixture, or at least 20% (v/v) of the resulting mixture, or at least 30% (v/v) of the resulting mixture, or at least 35% (v/v) of the resulting mixture, or at least 40% (v/v) of the resulting mixture, or at least 50% (v/v) of the resulting mixture, or at least 60% (v/v) of the resulting mixture, or at least 70% (v/v) of the resulting mixture, or at least 80% (v/v) of the resulting mixture, or at least 85% (v/v) of the resulting mixture, or at least 90% (v/v) of the resulting mixture, or at least 95% (v/v) of the resulting mixture, or at least 98% (v/v) of the resulting mixture, or at least 99% (v/v) of the resulting mixture.

The contact of the sample with the aqueous compositions can be done at a temperature ranging from 2° C. to 40° C., more preferably from 4° C. to 40° C., and for at least 1 min, for at least 2 min, for at least 5 min, for at least 10 min, or for at least 15 min.

In some embodiments of the method for collecting a sample of the present invention, the sample is a sample as defined herein. In some preferred embodiments, the sample is a biological sample as defined herein. In some preferred embodiments, biological samples include anterior nasal and mid-turbinate nasal swabs, nasopharyngeal (NP) and oropharyngeal (OP) swabs, nasopharyngeal washes/aspirates or nasal aspirates, and broncho alveolar lavage (BAL) specimens. In more preferred embodiments, said specimens are obtained from individuals suspected of COVID-19. In other embodiments, the sample is an environmental sample as defined herein.

In some preferred embodiments, the sample is directly introduced into a collection device or a collection vessel comprising the composition according of the present invention.

In other preferred embodiments, the sample is collected using a swab and introducing it into a collection device or a collection vessel comprising the composition according of the present invention. In more preferred embodiments, the swab is discarded after rubbing it against the collection device or a collection vessel.

In some embodiments, the sample comprises at least one pathogen. In some embodiments, said at least one pathogen is a fungi, a bacteria, a parasite or a virus. In some embodiments, said at least one pathogen is a virus. In other embodiments said virus is a respiratory virus. In some embodiments, said respiratory virus is from the coronaviridae family. In more preferred embodiments, said virus is SARS-COV-2. Other non-respiratory viruses are also contemplated, such as HIV, HCV, HBV, HAV, HEV, parvovirus, West Nile Virus, Usutu, Chikungunya, Dengue, or Zika, among others. In some embodiments, the at least one pathogen is a bacteria. In some embodiments, the at least one pathogen is a fungi. In some embodiments, the at least one pathogen is a parasite. Thus, other pathogens, such as Babesia or Plasmodium, are also contemplated within the scope of the present invention.

In a further aspect the present invention also relates to a collection device or vessel comprising the aqueous compositions as described herein. The present invention also relates to a sample collection kit comprising a collection device or a collection vessel and the aqueous compositions as described herein. In some embodiments, said sample collection kit further comprises a swab, a curette, or a culture loop. In some preferred embodiments, the sample collection kit comprises a collection device or a collection vessel, the aqueous compositions described herein and a swab.

In a further aspect the present invention relates to the use of the composition as described herein to collect a sample suspected of containing a target nucleic acid. In preferred embodiments, the sample is as defined herein. In some preferred embodiments, the sample is a biological sample as defined herein. In some preferred embodiments, biological samples include anterior nasal and mid-turbinate nasal swabs, nasopharyngeal (NP) and oropharyngeal (OP) swabs, nasopharyngeal washes/aspirates or nasal aspirates, and broncho alveolar lavage (BAL) specimens. In more preferred embodiments, said specimens are obtained from individuals suspected of COVID-19.

In some embodiments, the sample comprises at least one pathogen. In some embodiments, said at least one pathogen is a fungi, a bacteria, a parasite or a virus. In some embodiments, said at least one pathogen is a virus. In other embodiments said virus is a respiratory virus. In some embodiments, said respiratory virus is from the coronaviridae family. In more preferred embodiments, said virus is SARS-COV-2. Other non-respiratory viruses are also contemplated, such as HIV, HCV, HBV, HAV, HEV, parvovirus, West Nile Virus, Usutu, Chikungunya, Dengue, or Zika, among others. In some embodiments, the at least one pathogen is a bacteria. In some embodiments, the at least one pathogen is a fungi. In some embodiments, the at least one pathogen is a parasite. Thus, other pathogens, such as Babesia or Plasmodium, are also contemplated within the scope of the present invention.

### EXAMPLES

It should be readily apparent to one of ordinary skill in the art that the examples disclosed herein below represent generalised examples only, and that other compositions and methods capable of reproducing the invention are possible and are embraced by the present invention.

The following table summarizes exemplary compositions of the present invention used in the examples below.

TABLE 1
Exemplary compositions of the present invention

| | Composition C1 | | Composition C2 | | Composition C3 | |
|---|---|---|---|---|---|---|
| HEPES | 20% | (w/v) | 10% | (w/v) | 20% | (w/v) |
| LLS | 10% | (w/v) | 5% | (w/v) | 10% | (w/v) |
| Succinic acid | 3% | (w/v) | 1.4% | (w/v) | 3% | (w/v) |
| LiOH | 3% | (w/v) | 1.3% | (w/v) | 1.9% | (w/v) |

| | | | |
|---|---|---|---|
| pH | 7.4-7.5 | 7.2-7.4 | 6.15 |

The samples analyzed in the examples of the present invention can be considered valid/invalid and/or reactive/non-reactive. A sample is considered valid when it is validly processed by the testing system, which involves the sample being successfully pipetted by Procleix Xpress system, introduced into Procleix Panther system where an RNA based internal control (IC) is added and analyzed using the Procleix SARS-COV-2 assay or any other Procleix assay (Grifols Diagnostic Solutions Inc., USA) giving a positive result for the added IC. A valid sample can be either reactive (positive for SARS-COV-2 or other pathogen) or not reactive (negative for SARS-COV-2 or other pathogen) depending on the presence or absence of the target nucleic acid of interest. Sensitivity, which is the proportion of true positives correctly identified as such (e.g. the percentage of infected patients correctly identified as having the infection), is also indicated when relevant.

### Example 1: Comparison of Compositions of the Present Invention for Collecting Swab Samples

One of the purposes of the compositions of the present invention is to be used for swab specimen collection. Swab samples have a high viscosity, which in some cases makes their processing by the testing systems more difficult than for other biological liquid samples, such as serum or plasma. For investigating the suitability of the compositions of the present invention to collect such specimens and be further tested, nasal swab samples were collected from healthy volunteers (tested negative for SARS-COV-2) in compositions C1 and C3 (Table 1), and then processed on the Panther System using Procleix SARS-COV-2 assay according to the manufacturer's instructions. Compositions C1 and C3 with different LLS concentration (ranging from 3% to 10% (w/v)) were also tested.

Thus, the results shown in Table 2 demonstrate that compositions C1 and C3 are suitable for collecting swab samples as all samples resulted valid. In addition, said results also demonstrate that different concentrations of detergent and other components of the composition, in particular LiOH, do not affect the ability of said compositions to be used as collecting medium as all compositions tested resulted valid for collecting the specimens and being further analyzed in Panther.

TABLE 2
Validity of the compositions of the present invention to collect
nasal swab samples and being processed on the Panther System

| Composition | N | # Valid | # Invalid |
|---|---|---|---|
| 3% LLS C1 | 6 | 6 | 0 |
| 5% LLS C1 | 6 | 6 | 0 |
| 7% LLS C1 | 6 | 6 | 0 |
| 9% LLS C1 | 6 | 6 | 0 |
| 10% LLS C1 | 6 | 6 | 0 |
| 3% LLS C3 | 6 | 6 | 0 |
| 6% LLS C3 | 6 | 6 | 0 |
| 8% LLS C3 | 6 | 6 | 0 |

N: number of samples

In a further experiment, two new nasal swab collected samples on 6% LLS C3 were spiked with known quantities of artificial virus (50 μL of AccuPlex SARS-COV-2 (Seracare, ref 0505-0129) (61 copies/ml)) and run into the Panther system following Procleix SARS-COV-2 assay instructions. For both of them, reactive results were obtained.

In conclusion, the compositions of the present invention have been proved suitable for collecting biological samples comprising nucleic acids, even if said samples are nasal specimens, and suitable to be tested by molecular diagnostic methods and to detect nucleic acid of interest.

### Example 2: Comparison of VTM and Compositions of the Present Invention for Specimen Collection

The objective of this study was to evaluate the sensitivity of collecting nasal specimens using the compositions of the present invention and their stability over time. VTM was used for comparison, as it is the standard medium for swab collection.

VTM was prepared according to the recommendation of Centers for Disease Control and Prevention (SOP #: DSR-052-05). Briefly, 10 ml of inactivated fetal bovine serum (FBS) were added to a bottle of 500 ml of sterile Hanks Balanced Salt Solution (HBSS), followed by 2 mL of a Gentamicin/Amphotericin B mixture. This VTM was used for all the experiments conducted.

For a first comparative study, swabs from nasal specimens were collected and spiked with known quantities of artificial virus before being introduced into collection tubes comprising either 3 mL of VTM or 3 mL of composition C2 (Table 1). In particular, swabs from nasal specimens were collected from 17 healthy volunteers (tested negative for SARS-COV-2) using flocked swabs. Each swab containing the specimen was spiked with known quantities of artificial virus (3 μL or 7.5 μL of Member 1 (100 copies/μl) of AccuPlex SARS-COV-2 (Seracare, ref 0505-0129)) to obtain spiked swabs containing 0 copies/mL, 100 copies/mL or 250 copies/mL. Then, the spiked swabs were immersed in the tube and rubbed over the tube wall during 15 seconds followed by the discarding of the swab. All the samples were tested in triplicate within the next two hours after collection.

VTM collected specimens were processed following Procleix SARS-COV-2 assay instructions (GDSS-IFU-000047-EN v. 4.0), which involved vortexing of the collection tube followed by the transfer to a secondary tube containing an extraction buffer before the loading of the sample into the Panther System, whereas the specimens collected in composition C2 were directly loaded into the Panther System, just after taken off the cap (no swab inside).

The results for the negative controls (0 copies/mL) and the samples spiked with 100 and 250 copies/mL are shown in Table 3. The samples resulting positive for SARS-COV-2 were considered reactive, while a valid sample implies that the sample was suitable processed by the Panther system.

TABLE 3
Sensitivity using VTM and C2 composition

| | # | Total | | Re- | % Re- |
|---|---|---|---|---|---|
| Condition | Samples | replicates | Valid | active | active |
| Negative controls (VTM) | 2 | 6 | 6 | 0 | 0% |
| Negative controls (C2) | 3 | 9 | 9 | 0 | 0% |
| C2 100 copies/mL | 3 | 9 | 9 | 8 | 89% |
| VTM 100 copies/mL | 3 | 9 | 9 | 7 | 78% |
| C2 250 copies/mL | 3 | 9 | 9 | 9 | 100% |
| VTM 250 copies/mL | 3 | 9 | 9 | 9 | 100% |

The Limit of Detection (LOD) using composition C2 for sample collection was comparable to the current standard VTM collection. In fact, the % reactive in C2 was higher for spike corresponding to 100 copies/mL, taking into account that the LOD when the spike is done directly into VTM is 80 copies/mL. Since the LOD resulted comparable for the tested concentrations, a second experiment was carried out using same concentrations.

In a second experiment, swabs from nasal specimens were collected into composition C2 and then spiked with 750 and 300 copies of artificial virus Accuplex SARS-COV-2 to obtain 250 copies/mL and 100 copies/mL respectively in order to test stability of the sample over time. As control, swabs from nasal specimens were also collected into VTM and spiked with 300 copies (100 copies/mL).

In particular, nasal swabs from 37 healthy volunteers (tested negative for SARS-COV-2) were collected using flocked swabs. From these, a set of 10 swab specimens collected in VTM (experiment control) were spiked with 300 copies to obtain 100 copies/mL. The other 27 swab specimens were collected in composition C2, and from these, 10 swab specimens were spiked with 300 copies to obtain 100 copies/mL, 10 swab specimens were spiked with 750 copies to obtain 250 copies/mL and 6 swab specimens were spiked with 0 copies (negative control). All samples were tested after 2-3 hours from being collected (t0) by duplicate, and stored at RT for 48 hours (T48) for being re-tested once more, with no additional mixing. The samples were processed in Panther as previously explained. Results are shown in Table 4.

TABLE 4
Preservation of swab samples

| | | Negative | C2 | C2 | VTM |
|---|---|---|---|---|---|
| | | controls | 100 | 250 | 100 |
| Condition | | (C2) | copies/mL | copies/mL | copies/mL |
| # Samples | | 6 | 10 | 10 | 10 |
| T0 | Total replicates | 12 | 20 | 20 | 20 |
| | Valid | 12 | 20 | 20 | 20 |
| | Reactive | 0 | 20 | 20 | 20 |
| | % Reactive | 0% | 100% | 100% | 100% |
| T48 | Total replicates | 6 | 10 | 1 | 10 |
| | Valid | 5 | 10 | 10 | 10 |
| | Reactive | 0 | 10 | 10 | 10 |
| | % Reactive | 0% | 100% | 100% | 100% |

The comparative experiment results confirmed the comparable LOD for both collection methods, and also sensitivity comparable to the previous studies using Accuplex directly spiked into the current standard VTM (80 copies/mL).

After 48 hours of collection, all replicates were reactive. Therefore, considering that the comparison with the current method (VTM) resulted in similar percentage of reactivity and all results showed a similar LOD, it can be concluded that the collection method does not decrease the sensitivity of the SARS-COV-2 detection in a significant manner. However, the collection method using the compositions of the present invention reduces the number of steps and improves sample traceability during the preparation of the sample as it is no longer necessary to transfer the sample to a secondary tube containing an extraction buffer before the loading of the sample into the Panther System. Thus, the composition of the present invention is suitable for both collection of the specimen and preservation of the nucleic acid until the molecular testing.

A further experiment was conducted to evaluate preservation of SARS-COV-2 RNA at room temperature for longer periods. In particular, swabs from nasal specimens were collected from 3 healthy volunteers (tested negative for SARS-COV-2) using flocked swabs. Each swab containing the specimen was spiked with known quantities of artificial virus (100, 250 y 500 copies/ml of Accuplex (Seacare). Then, the spiked swabs were immersed in a tube comprising 3 ml of composition C2, rubbed over the tube wall and bottom during 15 seconds followed by the discarding of the swab. All the samples were tested in triplicate just after collection. The specimens were directly loaded of the sample into the Panther System or stored at room temperature for 7 days.

The results showed that all the tested samples were reactive after 7 days of storage at room temperature, which confirms that the composition of the present invention preserves RNA also at room temperature for at least 7 days.

### Example 3: Evaluation of Viral Inactivation by the Compositions of the Present Invention

The ability of composition C2 to inactivate SARS-COV-2 was evaluated by titration assays on Vero cells (Pasteur Institute. Simizu B. and Terasima T. 1988; Simizu B. et al., 1967).

The titration method is a quantitative assay in which the virus titer measurement is based on the detection of virus production in the infected cells, by observation of a specific cytophatic effect. In order to evaluate the inactivation capacity of the compositions of the present invention, said compositions, in particular C2, was spiked with SARS-COV-2 at a 1/10 ratio at room temperature and incubated for 2 min, 10 min or 30 min (test samples T1, T2 and T3 respectively). As positive control, the virus was spiked in stock medium (V1).

After incubation at room temperature, 0.5 mL of each sample in triplicate were diluted with dilution medium and ultracentrifuged. Pellet was resuspended in 25 mL of titration medium and immediately titrated with Vero cells. Ultracentrifugation was shown not to have a significant effect on the virus.

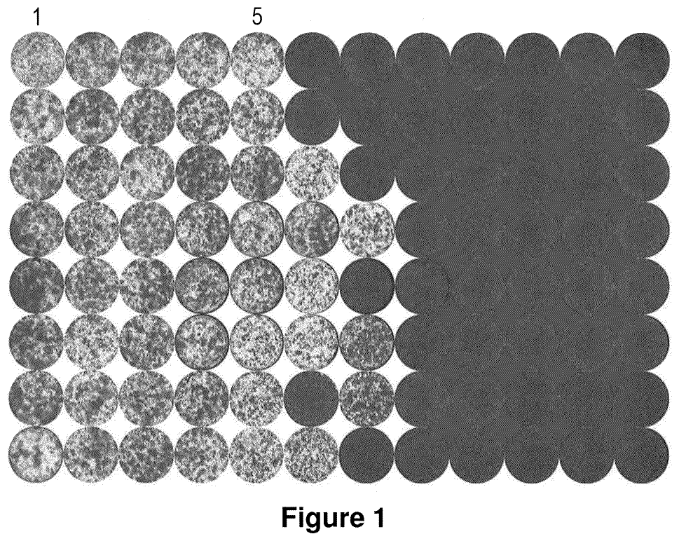

Test samples were then diluted with medium by serial 3-fold dilutions (eight replicates for each dilution) across a 96 well plate (“sample dilution plate”). Each well from the “sample dilution plate” was then inoculated on the corresponding well of a new plate (“sample titration plate”). Cell suspension was added to each well of the “sample titration plate” and the plates were then incubated at appropriate temperature. After a period of incubation allowing viral replication and infection of adjacent cells, wells with foci were counted after infection by observation under inverted light microscope or a stain overlay (crystal violet) was added and wells are examined for cytopathic effect. The infected wells show up as clear areas whereas the non-infected wells are stained (FIG. 1).

The infectious titer [m(T)], expressed as 50% tissue culture infective dose per milliliter (TCID50/mL), was calculated using the Spearman-Karber formula. The virus load, defined by its mean value [m(L)] and its confidence interval, was calculated as the mean titer m(T)×Vt, where Vt is the total volume of sample, that is 1 ml for all cases. The virus reduction factor (R), defined as the Log 10 of the ratio of the virus load (Li) in the pre-treatment material (non-treated) and the virus load (Lf) in the post-treatment material, is calculated as [m(R)]=Log 10 (V1)−Log₁₀ (LS), being LS the virus load of each sample T1, T2 or T3. Results are shown in Table 5.

TABLE 5
Virus inactivation by the compositions of the present invention

| | | | | | Reduction factor |
|---|---|---|---|---|---|
| | | | | | evaluation R |
| | Infections Titer (T) | | | | Mean reduction |
| | Mean titer [m(T)] | | | | factor m(R) and |
| | and confidence | | Virus Load (L) | | confidence |
| Sample | interval | | Mean load | | interval |
| V1 (positive | 6.07 | 5.85 ≤ | 6.07 | 5.85 ≤ | NA |
| control) | | T ≤ 6.29 | | T ≤ 6.29 | |
| T1 (T0 + | 0.98 | NA | 0.98 | NA (no | Run a > 5.09 |
| 2 min) | | | | virus) | Run b > 5.09 |
| | | | | | Run c > 5.09 |
| T2 (T0 + | 0.98 | NA | 0.98 | NA (no | Run a > 5.09 |
| 10 min) | | | | virus) | Run b > 5.09 |
| | | | | | Run c > 5.09 |
| T3 (T0 + | 0.98 | NA | 0.98 | NA (no | Run a > 5.09 |
| 30 min) | | | | virus) | Run b > 5.09 |
| | | | | | Run c > 5.09 |

The resulted virus reduction factor (R) for composition C2, for all the times tested in this study (2 min, 10 min and 30 min), was higher than 5.09. Therefore, a reduction titer of more 5 Log (99.999%) of SARS-COV-2 was demonstrated for the composition of the present invention.

### Example 4. Inactivation of Viral Nucleic Acids of Other Viruses than SARS-COV-2

Clinical HIV-1, HIV-2, HCV, HBV and HEV plasma and serum positive specimens (with positive serological and/or NAT result) were obtained from American Red Cross (Gaithersburg, MD), Japanese Red Cross (Tokyo, Japan), Boca Biolistics (Pompano Beach, FL), Medical Research Network (MRN) (New York, NY), SlieaGen (Austin, TX), Bioreclamation (Westbury, NY), Cerba (France), Discovery Live Sciences (Los Osos, CA) and Access Biologicals (San Diego, CA).

Plasma samples: One hundred HIV-1, HCV, and HBV, 100 HEV, and 37 HIV-2 positive plasma specimens were tested neat in singlet using the Procleix UltrioPlex E assay (UPE). The same set of specimens was tested in singlet in pool of 16 and in pool of 96. Pooled samples were created by mixing 1 part of positive specimen with 1 part from 15 (for pool of 16) or 95 (for pool of 96) different plasma normal donor specimens.

Serum samples: Twenty five HIV-1, HIV-2, HCV, and HBV positive serum specimens were tested neat in singlet using the UPE assay. The same set of specimens was tested in singlet in pool of 16. Pooled samples were created by mixing 1 part of positive specimen with 1 part from 15 different normal serum donor specimens.

The liquid samples were diluted in composition C1 in a ratio 0.75:1 (C1:sample) during the first step of the assay.

TABLE 6
Virus inactivation in plasma samples

| | Sample (Viral Load) | N | TP | FN | Sensitivity | 95% CI |
|---|---|---|---|---|---|---|
| Neat | HIV-1 (≥100 c/mL) | 99 | 99 | 0 | 100.0 | 96.3-100.0 |
| | HIV-1 (≥10 c/mL) | 100 | 100 | 0 | 100.0 | 96.4-100.0 |
| | HIV-2 (≥100 c/mL) | 18 | 18 | 0 | 100.0 | 81.5-100.0 |
| | HCV (≥100 c/mL) | 100 | 100 | 0 | 100.0 | 96.4-100.0 |
| | HBV (≥100 c/mL) | 91 | 91 | 0 | 100.0 | 96.0-100.0 |
| | HBV (≥10 c/mL)* | 100 | 100 | 0 | 100.0 | 96.4-100.0 |
| | HEV (≥100 c/mL) | 73 | 73 | 0 | 100.0 | 95.1-100.0 |
| | HEV (≥1 c/mL) | 100 | 100 | 0 | 100.0 | 96.4-100.0 |
| Diluted | HIV-1 (≥100 c/mL) | 99 | 99 | 0 | 100.0 | 96.3-100.0 |
| (1:16) | HIV-1 (≥10 c/mL) | 100 | 99 | 1 | 99.0 | 94.6-100.0¹ |
| | HIV-2 (≥100 c/mL) | 18 | 18 | 0 | 100.0 | 81.5-100.0 |
| | HCV (≥100 c/mL) | 100 | 100 | 0 | 100.0 | 96.4-100.0 |
| | HBV (≥100 c/mL) | 91 | 91 | 0 | 100.0 | 96.0-100.0 |
| | HBV (≥10 c/mL)* | 100 | 97 | 3 | 97.0 | 91.5-99.4 |
| | HEV (≥100 c/mL) | 73 | 73** | 0 | 100.0 | 95.1-100.0 |
| | HEV (≥1 c/mL) | 100 | 87 | 13 | 87.0 | 78.8-92.9 |
| Diluted | HIV-1 (≥100 c/mL) | 99 | 98 | 1 | 99.0 | 94.5-100.0¹ |
| (1:96) | HIV-1 (≥10 c/mL) | 100 | 98 | 2 | 98.0 | 93.0-99.8 |
| | HIV-2 (≥100 c/mL) | 18 | 15 | 3 | 83.3² | 58.6-96.4 |
| | HCV (≥100 c/mL) | 100 | 100 | 0 | 100.0 | 96.4-100.0 |
| | HBV (≥100 c/mL) | 91 | 85 | 6 | 93.4³ | 86.2-97.5 |
| | HBV (≥10 c/mL)* | 100 | 87 | 13 | 87.0 | 78.8-92.9 |
| | HEV (≥100 c/mL) | 73 | 68 | 5 | 93.2⁴ | 95.1-100.0 |
| | HEV (≥1 c/mL) | 100 | 77 | 23 | 77.0 | 67.5-84.8 |

N = Number of specimen,
TP = True Positive,
FN = False Negative,
CI = Clopper-Pearson Confidence Interval
*N = 5 with viral load indicated as <20 c/mL
**One HEV positive sample was HIV/HCV/HBV reactive when tested diluted 1:16, but HIV/HCV/HBV non-reactive when tested neat or tested diluted 1:96.
¹Exact Upper limit = 99.97
²Sensitivity was 100% (1/1) for samples ≥100 c/mL after dilution
³Sensitivity was 100% (54/54) for samples ≥100 c/mL after dilution
⁴Sensitivity was 100% (40/40) for samples ≥100 c/mL after dilution

TABLE 7
Virus inactivation in serum samples

| Test | | | | | | |
|---|---|---|---|---|---|---|
| Level | Sample (Viral Load) | N | TP | FN | Sensitivity | 95% CI |
| Neat | HIV-1 (≥100 c/mL) | 25 | 25 | 0 | 100.0 | 86.3-100 |
| | HIV-2 (≥100 c/mL) | 7 | 7 | 0 | 100.0 | 59.0-100.0 |
| | HCV (≥100 c/mL) | 25 | 25 | 0 | 100.0 | 86.3-100 |
| | HBV (≥100 c/mL) | 25 | 25 | 0 | 100.0 | 86.3-100 |
| Diluted | HIV-1 (≥100 c/mL) | 25 | 25 | 0 | 100.0 | 86.3-100 |
| (1:16) | HIV-2 (≥100 c/mL) | 7 | 7 | 0 | 100.0 | 59.0-100.0 |
| | HCV (≥100 c/mL) | 25 | 25 | 0 | 100.0 | 86.3-100 |
| | HBV (≥100 c/mL) | 25 | 25 | 0 | 100.0 | 86.3-100 |
| | HEV (≥100 c/mL) | 26 | 26 | 0 | 100.0 | 86.8-100.0 |

N = Number of specimen,
TP = True Positive,
FN = False Negative,
CI = Clopper-Pearson Confidence Interval

For serum (table 6) and plasma samples (table 7), the high sensitivity obtained (up to 1 copy/ml of virus) demonstrated the lysis of the samples, as lysis is needed for detection of the virus. This demonstrates that the composition of the present invention is suitable for inactivation of other virus in liquid samples.

### Example 5. Inactivation and Preservation of SARS-COV-2 RNA

The purpose of a first experiment was to evaluate inactivation of SARS-COV-2 by the compositions of the present invention and preservation during storage. For that, the following samples were prepared:
Contrived SeraCare positive samples were prepared by spiking SARS-COV-2 positive material at 123 copies/mL to the CDC recommended viral transport medium (VTM). The spiked concentration corresponded to about 3×LOD for SCV2 Assay for the swab specimen type.
Contrived BEI SARS-Related Coronavirus samples were prepared by spiking Heat inactivated positive material at 63.87 c/mL to the CDC recommended viral transport medium (VTM).

Liquid samples were then mixed 1:1 with composition C1 and stored i) at 30° C. and tested at 0 hours, 60 hours, 90 hours 8 days and 17 days, or ii) at 4° C. and tested at 0 hours, 8 days and 17 days. Results for i) and ii) are shown in tables Table 8 and 9 respectively.

TABLE 8
Results for samples stored at 30° C.

| Spiking | Time | | | % | % | Analyte RLU | | | IC RLU | | |
|---|---|---|---|---|---|---|---|---|---|---|---|
| Material | Point | | N | Valid | Reactive | Average | SD | % CV | Average | SD | % CV |
| SeraCare | Baseline | | 30 | 100% | 100% | 1,222,664 | 33,229 | 2.72% | 177,578 | 29,575 | 16.65% |
| | 60 | hours | 20 | 100% | 100% | 1,171,150 | 27,282 | 2.33% | 218,477 | 19,334 | 8.85% |
| | 90 | hours | 20 | 100% | 100% | 1,180,477 | 37,500 | 3.18% | 203,306 | 23,228 | 11.43% |
| | 8 | days | 20 | 100% | 100% | 1,220,246 | 19,424 | 1.59% | 220,597 | 17,340 | 7.86% |
| | 17 | days | 20 | 100% | 100% | 1,204,841 | 22,612 | 1.88% | 181,007 | 21,217 | 11.72% |
| BEI | Baseline | | 60 | 100% | 100% | 1,306,134 | 43,935 | 3.36% | 194,046 | 40,063 | 20.65% |
| Resources | 60 | hours | 20 | 100% | 100% | 1,279,460 | 50,627 | 3.96% | 206,686 | 38,576 | 18.66% |
| | 90 | hours | 20 | 100% | 100% | 1,303,896 | 45,285 | 3.47% | 181,427 | 38,596 | 21.27% |
| | 8 | days | 20 | 100% | 100% | 1,268,492 | 60,598 | 4.78% | 226,171 | 50,195 | 22.19% |

SD = standard deviation,
% CV = percent coefficient of variance

TABLE 9
Results for samples stored at 4° C.

| Spiking | Time | | | % | % | Analyte RLU | | | IC RLU | | |
|---|---|---|---|---|---|---|---|---|---|---|---|
| Material | Point | | N | Valid | Reactive | Average | SD | % CV | Average | SD | % CV |
| SeraCare | Baseline | | 30 | 100% | 100% | 1,222,664 | 33,229 | 2.72% | 177,578 | 29,575 | 16.65% |
| | 8 | days | 20 | 100% | 100% | 1,223,982 | 26,525 | 2.17% | 222,972 | 19,116 | 8.57% |
| | 17 | days | 20 | 100% | 100% | 1,204,841 | 22,612 | 1.88% | 181,007 | 21,217 | 11.72% |
| BEI | Baseline | | 60 | 100% | 100% | 1,306,134 | 43,935 | 3.36% | 194,046 | 40,063 | 20.65% |
| Resources | 8 | days | 30 | 97% | 100% | 1,293,862 | 48,564 | 3.75% | 190,639 | 41,382 | 21.71% |

Seracare processed swab specimen samples spiked to 123 copies/mL of SARS-COV-2 positive material were 100% reactive at baseline and after storage at 30° C.±3° C. for 60 hours, 90 hours, 8 days, and 17 days. Swab specimen stored in 4° C.±3° C. were 100% reactive at baseline and after 8 days and 17 days.

BEI Heat Inactivated Processed Swab Specimen samples spiked to 63.87 copies/mL of SARS-CoV-2 positive material were 100% reactive at baseline and after storage at 30° C.±3° C. for 60 hours, 90 hours, and 8 days. Swab specimens stored in 4° C.±3° C. were 100% reactive at baseline and after 8 days.

Although the RNA spiked is these experiments is artificial capside (Seracare sample) or real capside (BEI inactivated virus), it was already demonstrated in example 4 that the composition of the present invention is able to lyse the samples and preserve RNA even at 30° C.

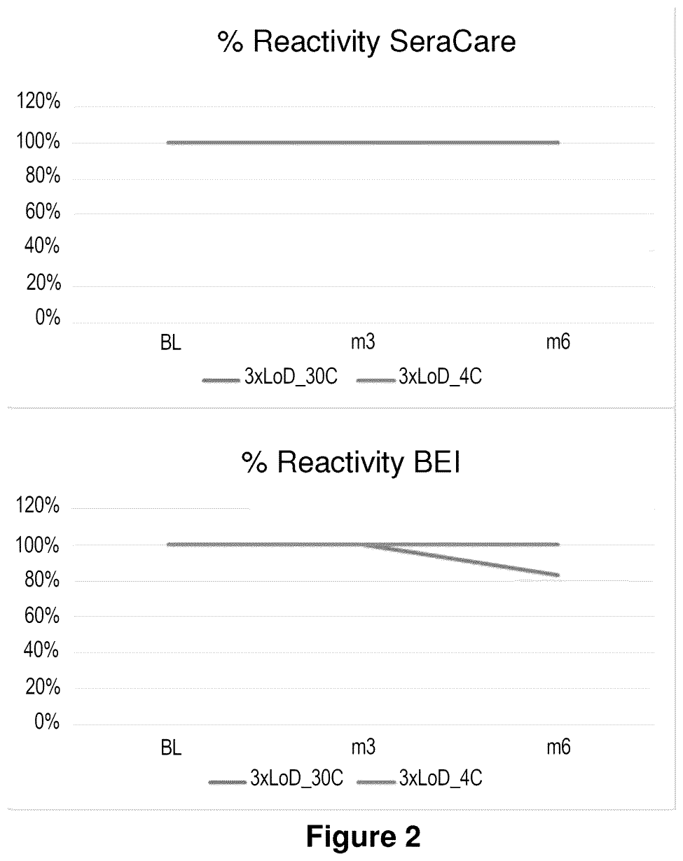

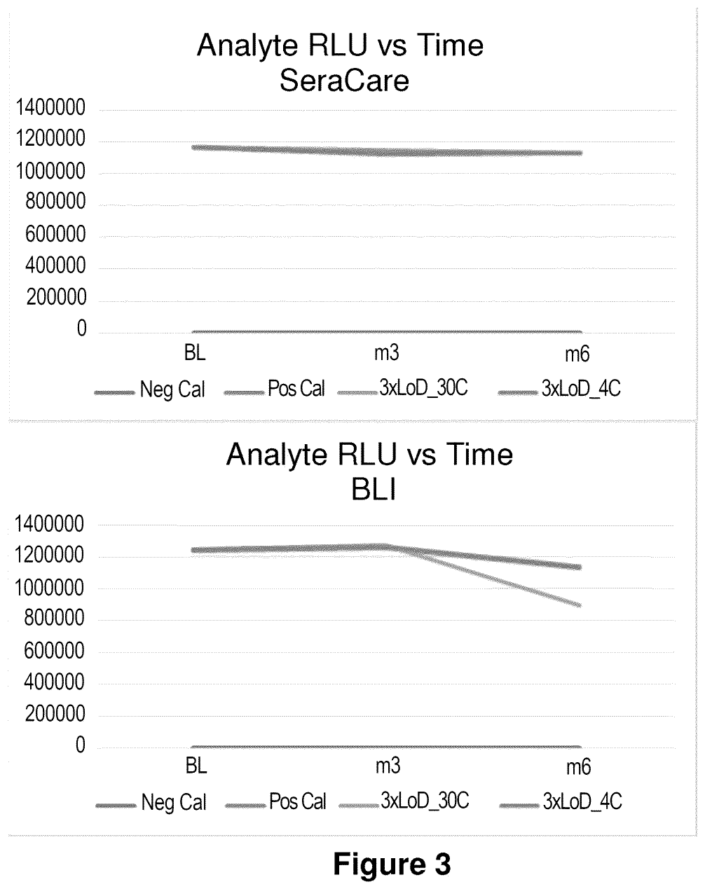

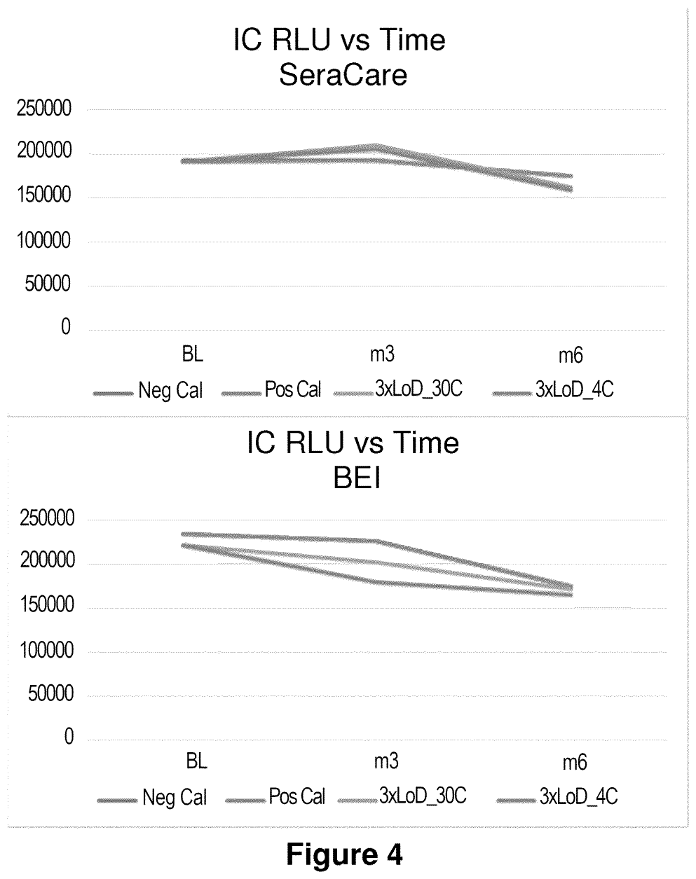

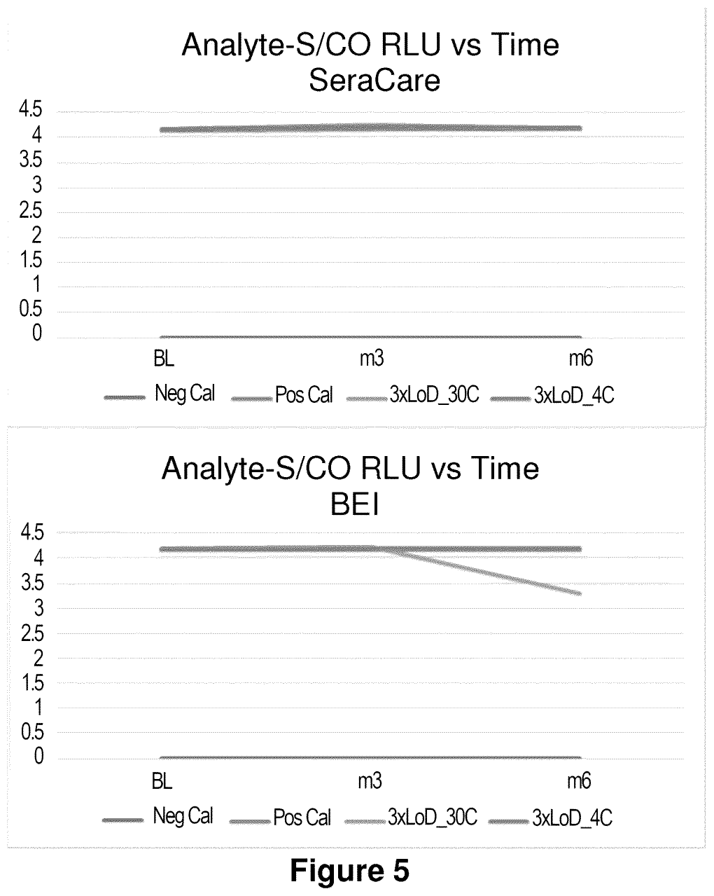

In a second experiment, the previous samples (SeraCare RNA (SC) or Heat-inactivated-virus (BEI) spiked at 3×LOD in VTM, followed by adding C1 at 1:1) were stored at either 30° C. or 4° C. for longer periods, and then re-tested at 3 months and 6 months following the same Panther protocol. The results demonstrated that SeraCare RNA is stable at both 4° C. and 30° C. storages for at least 6 months while BEI is stable at 4° C. storage for at least 6 months. BEI at 30° C. storage was stable for at least 3 months. The related results are shown in FIGS. 2 to 5.

In another experiment, the preservation of SARS-COV-2 real positive samples was also studied. For that, 35 nasopharyngeal samples collected in composition C2 from volunteers tested positive for SARS-COV-2, were re-tested after being stored at temperature between 2° C. and 8° C. for up to 18 days. All the tested samples were reactive (positive) after testing in Panther which demonstrates that RNA is preserved also at cold temperatures up to 18 days.

### Example 6. Long-Term Preservation of Pathogen RNA

For this experiment, the following samples were prepared:
In vitro synthetized transcript (IVT) for Babesia 18s ribosomal RNA at 500 c/mL diluted in composition C1 for obtaining 500, 100 or 30 copies/mL
Control: IVT for an HIV-1 scrambled RNA sequences not found in any organisms diluted in composition C1

Babesia IVT and control were stored at different temperatures and then tested for the presence of the target RNA using the Babesia Procleix assay and Panther system (Grifols Diagnostic Solutions Inc., USA).

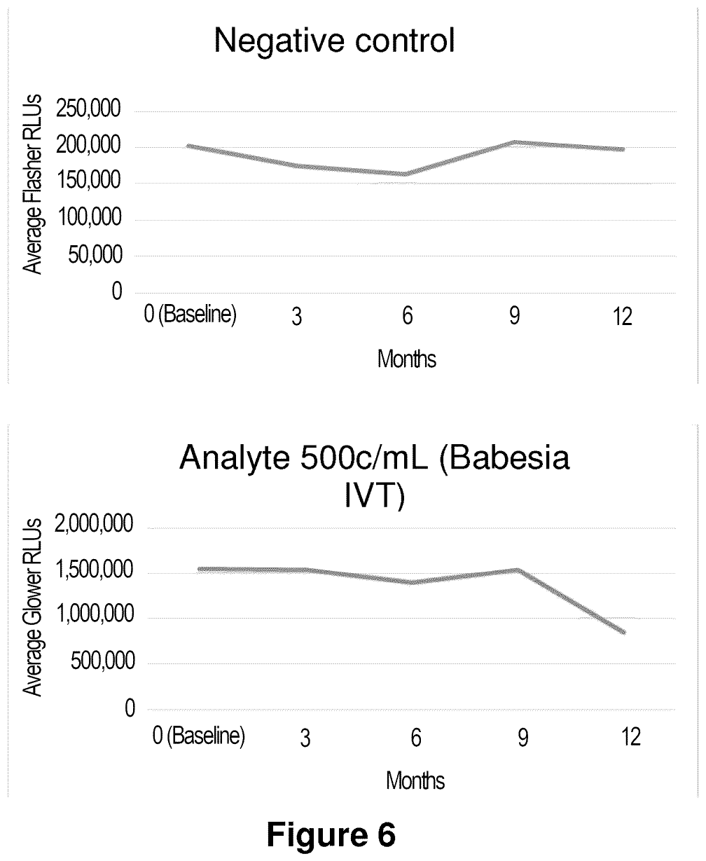

In the first experiment, all samples were stored at 5° C.−/+3° C. instead of their intended storage temperature of −15° C. to −35° C. The samples were then tested at 0, 3, 6, 9, and 12 months. The results, shown in FIG. 6, demonstrate that the composition of the present invention allows preservation of pathogen RNA for at least 9 months at 5° C.

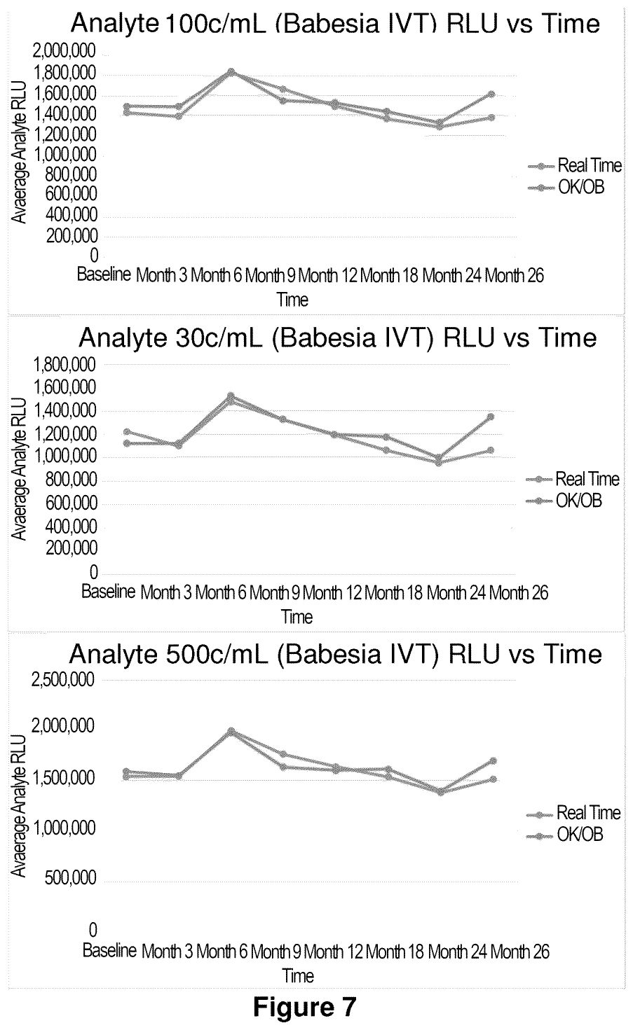

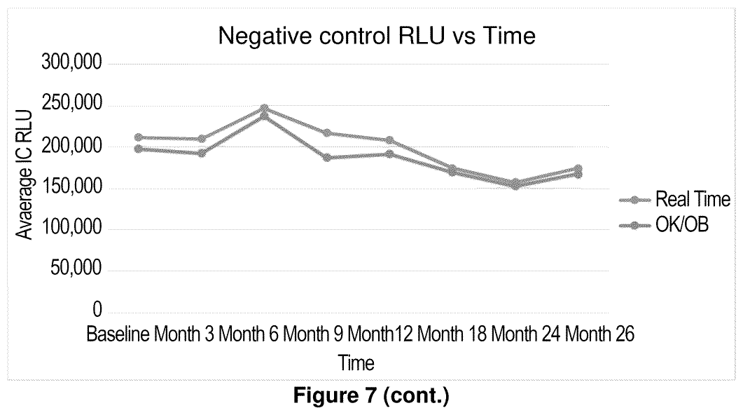

In another experiment, the samples were stored at −20° C.−/+5° C., their intended storage temperature. The samples were then tested at 0, 3, 6, 9, 12, 18, 24 and 26 months. At each time point, testing was done the day of the kit was opened and 36 days after opening after being placed on the Panther system for 72 hours (OB for on-board). The results, shown in FIG. 7, demonstrate that the composition of the present invention allows preservation of pathogen RNA for at least 26 months at −20° C., even with high content of RNA.

In conclusion, the compositions of the present invention are suitable for collection of samples, such as biological, environmental or waste samples, and in particular for collection of respiratory swab samples as demonstrated in examples 1 and 2 or liquid samples as demonstrated in example 4. Said compositions are also an effective collecting medium instead of the current protocol using VTM, which inactivates the potential pathogens making the sample safer for the personal working with it (avoiding Safety Cabinet use for pathogens like SARS-COV-2), reduce the steps for preparation of a sample for nucleic acid testing as the same collection medium is able to lyse and inactivate the pathogens present in the sample (as shown in examples 3, 4 and 5) and at the same time it successfully preserve the integrity of the nucleic acids until testing. In particular, the present compositions have been proved suitable for preserving sample comprising nucleic acids during storage at temperatures ranging from 4° C. to 30° C. from a few hours up to 9 months (see examples 5 and 6) and when stored at −20° C. up to 26 months (see example 6). Finally, the composition of the present invention is less harmful than other available inactivation mediums, which are often based in guanidine thiocyanate, and it is also compatible with testing systems using sodium hypochlorite during their cleaning processes.

### Example 7: Comparison of Compositions of the Present Invention Comprising Foam Ban for Collecting Swab Samples

Nasal swab samples were collected from healthy volunteers (tested negative for SARS-COV-2) in compositions C1 and C3 (Table 1) with different concentrations of LLS (ranging from 3% to 9% (w/v)) and comprising Foam Ban at a concentration of 300 μl/L and then processed on the Panther System using Procleix SARS-COV-2 assay according to the manufacturer's instructions. Samples were collected using two different commercial swabs: Aptima® Multitest Swab Specimen Collection Kit (Hologic Inc, USA) and ViCUM® Flocked (Deltalab, Spain).

All samples were validly processed, as shown in Table 10, demonstrating that compositions C1 and C3 comprising Foam Ban are suitable for collecting swab samples. It was also demonstrated that when compositions comprise different concentrations of detergent and other components of the composition, in particular LiOH, do not affect the ability of said compositions to be used as collecting medium either, as all compositions tested resulted valid for collecting the specimens and being further analyzed in Panther.

TABLE 10
Validity of the compositions of the present invention
comprising Foam Ban to collect nasal swab samples
and further processing on the Panther System.

| Swab Type | Composition | N | #Valid | #Invalid |
|---|---|---|---|---|
| Aptima | 3% C1 | 4 | 4 | 0 |
| Multitest | 5% C1 | 2 | 2 | 0 |
| | 7% C1 | 2 | 2 | 0 |
| | 9% C1 | 2 | 2 | 0 |
| | 3% C3 | 4 | 4 | 0 |
| | 6% C3 | 2 | 2 | 0 |
| | 8% C3 | 2 | 2 | 0 |
| ViCUM | 3% C1 | 8 | 8 | 0 |
| Flocked | 5% C1 | 4 | 4 | 0 |
| | 7% C1 | 4 | 4 | 0 |
| | 9% C1 | 4 | 4 | 0 |
| | 3% C3 | 8 | 8 | 0 |
| | 6% C3 | 4 | 4 | 0 |
| | 8% C3 | 4 | 4 | 0 |

In a further experiment, Nasal swab samples were collected from healthy volunteers (tested negative for SARS-COV-2) in compositions C1 and C3 (Table 1) with different concentrations of LLS (ranging from 3% to 10% (w/v)) and comprising Foam Ban at a concentration of 300 μl/L. Samples were kept at 4° C. for 6 days before being processed on the Panther System using Procleix SARS-COV-2 assay according to the manufacturer's instructions. Samples were collected using two different commercial swabs: Aptima® Multitest Swab Specimen Collection Kit (Hologic Inc, USA) and ViCUM® Flocked (Deltalab, Spain) As shown in Table 11, all samples were validly processed, demonstrating that compositions of the present invention comprising Foam Ban allow collection and storage of samples for at least 6 days at 4° C. before processing. Also in this case, it is demonstrated that different concentrations of detergent and other components of the composition, in particular LiOH, do not affect the ability of said compositions to be used as collecting medium.

TABLE 11
Validity of the compositions of the present invention comprising
Foam Ban to collect nasal swab samples and store at 4° C.
for at least 6 days and further processing on the Panther System.

| Swab Type | Composition | N | #Valid | #Invalid |
|---|---|---|---|---|
| Aptima | 3% C1 | 4 | 4 | 0 |
| Multitest | 5% C1 | 4 | 4 | 0 |
| | 3% C3 | 4 | 4 | 0 |
| | 6% C3 | 4 | 4 | 0 |
| ViCUM | 3% C1 | 8 | 8 | 0 |
| Flocked | 5% C1 | 8 | 8 | 0 |
| | 7% C1 | 4 | 4 | 0 |
| | 10% C1 | 4 | 4 | 0 |
| | 3% C3 | 8 | 8 | 0 |
| | 6% C3 | 8 | 8 | 0 |
| | 8% C3 | 4 | 4 | 0 |

### Example 8: Inactivation and Preservation of SARS-COV-2 RNA Under Changing Storage Temperature Conditions

An experiment was carried out in order to test the inactivation and preservation of SARS-COV-2 RNA under temperature changing storage conditions.

A clinical matrix was prepared by pooling left-overs of nasal specimens collected from healthy volunteers (negative tested for SARS-COV-2). Each specimen had been collected immersing a nasal swab in a tube comprising 3 mL of composition C1 comprising Foam Ban at a concentration of 300 μl/L, diluted to 50% (v/v) with 0.9% (w/v) Sodium Chloride and rubbing the swab against the tube.

20 spiked samples of 3 mL at 2 times LoD (low positives, 160 copies per mL), 10 spiked samples at 5 times LoD (high positives, 400 copies per mL) and 10 negative samples to monitor for false positives, were prepared by spiking virus using recombinant virus containing SARS-COV-2 RNA (AccuPlex SARS-COV-2 from LGC SeraCare) onto clinical matrix.

TABLE 12
Sample panel description

| | | | | AccuPlex | Positive |
|---|---|---|---|---|---|
| | | Samples | Negative | (Member 1) | control |
| | Concen- | per pool | Pool | Volume | concen- |
| | tration | (3 mL | Volume | spiked | tration |
| Sample | (copies/mL) | aliquots) | (mL) | (μL) | (c/mL) |
| Negative | 0 | 10 | 32 | 0 | 0 |
| Low | 2 × LoD | 20 | 2 × 32 | 51.2 per pool | 160 |
| positive | (160 c/mL) | | | | |
| High | 5 × LoD | 10 | 32 | 128 | 400 |
| positive | (400 c/mL) | | | | |

The prepared 40 tubes containing 3 mL of sample according to the panel in table 12, were cycled through the temperatures and times according to the next workflow:
these samples were first kept at the temperature condition A (table 13)
at the end of the cycles these samples were analyzed with Procleix SARS-COV-2 assay in the Panther system
subsequently the same samples (2.5 mL each) were kept at the temperature condition B (table 14)
and after last cycle samples were analyzed with Procleix SARS-COV-2 assay in the Panther system.

TABLE 13
Temperature condition A

| | Cycle | Cycle Period | Total Time |
|---|---|---|---|
| Temperature | Period | Hours | Hours |
| 40° C. | 1 | 2 | 2 |
| RT | 2 | 16 | 18 |
| 40° C. | 3 | 8 | 26 |
| 25° C. | 4 | 21 | 47 |
| 40° C. | 5 | 6 | 53 |
| RT | 6 | 1 | 54 |

TABLE 14
Temperature condition B

| | | | |
|---|---|---|---|
| | Cycle | Cycle Period | Total Time |
| Temperature | Period | Hours | Hours |

| | | | | |
|---|---|---|---|---|
| −20° | C. | 1 | 72 | 72 |

| | | | |
|---|---|---|---|
| RT | 2 | 4 | 76 |

| | | | | |
|---|---|---|---|---|
| −20° | C. | 3 | 2 | 78 |
| 4° | C. | 4 | 40 | 118 |
| −20° | C. | 5 | 6 | 124 |

| | | | |
|---|---|---|---|
| RT | 6 | 3 | 127 |

All the runs resulted valid (Table 15), indicating that the compositions of the present invention are able to lyse the samples and preserve RNA even under changing storage temperature conditions.

TABLE 15
Results of testing with Procleix SARS-
CoV-2 assay in the Panther system:

| | | Total | | % |
|---|---|---|---|---|
| Condition | Specimen | samples | Reactive | Agreement |
| After | Negative | 10 | 0 | 100 |
| condition A | Low positive | 20 | 20 | 100 |
| | High positive | 10 | 10 | 100 |
| After | Negative | 10 | 0 | 100 |
| Condition A + | Low positive | 20 | 20 | 100 |
| Condition B | High positive | 10 | 10 | 100 |

### Example 9: Detection of Specimen-Derived Target Nucleic Acid

Nasal specimens from 42 healthy volunteers were collected using flocked swabs and immersing each swab in a tube comprising 3 mL of composition C1 comprising Foam Ban at a concentration of 300 μl/L, diluted to 50% (v/v) with 0.9% (w/v) Sodium Chloride. Swabs were rubbed over the tube wall and bottom during 15 seconds followed by the discarding of the swab, except for one tube where the swab was not discarded.

All the collected samples were stored 24-72 hours before being tested to determine the presence of human content and, therefore, to verify the correct collection of the specimen for further nucleic acid detection. The testing was done by detecting the human RNase P gene in negative control samples and in the collected clinical specimens. Samples were first extracted using QIAamp® MinElute® Virus Spin Kit, following manufacturer instructions, then evaluated using Nanodrop and finally tested for detecting human content using the CDC 2019-Novel Coronavirus (2019-nCOV) Real-Time RT-PCR Diagnostic Panel (IDT). This panel is designed for specific detection of SARS-COV-2 (two primer/probe sets) and this kit also includes an additional primer/probe set to detect the human RNase P gene, the assay that was carried out to detect human content in the samples.

All samples tested positive for human RNase P gene at Ct comparable to those obtained when analyzing fresh human samples of the same nature using other mediums, indicating that the composition of the present invention is suitable to collect and store a sample suspected of containing a target nucleic acid for further nucleic acid testing, regardless of the target nucleic acid being a pathogen-derived nucleic acid or a specimen-derived target nucleic acid.

### Example 10: Compositions of the Present Invention for Saliva Specimens Collection and Preservation

The objective of this study was to evaluate the compositions of the present invention as media for collecting and preservation of saliva specimens.

For a first experiment, 2.5 mL of saliva specimens from a healthy volunteer (tested negative for SARS-COV-2) were collected and introduced into collection tubes comprising 2.5 mL of composition C1 (Table 1) comprising Foam Ban at a concentration of 300 μl/L. The content of each collection tube was divided into two equal aliquots of 2.5 mL. One of the aliquots was spiked with a known quantity of heat-inactivated virus (7.5 μL of SARS-Related Coronavirus 2, Isolate USA-WA 1/2020; Catalog No. NR-52286, BEI Resources) to obtain a spiked sample at 30 copies/mL. The other aliquot was used as negative control (0 copies/mL). Both aliquots were tested in 4 replicates within the next two hours after collection by directly loading each replicate on the Panther System, immediately after the cap was removed. The results for the negative controls (0 copies/mL) and the spiked samples (30 copies/mL) are shown in Table 16. The samples showing positive results for SARS-COV-2 were considered reactive, while a valid sample implies that the sample was suitably processed by the Panther System.

TABLE 16
Sensitivity using C1 composition

| | # | # | | | |
|---|---|---|---|---|---|
| Condition | Samples | Replicates | Valid | Reactive | % Reactive |
| Negative controls | 1 | 4 | 4 | 0 | 0% |
| 30 copies/mL | 1 | 4 | 4 | 4 | 100% |

This experiment shows that the compositions of the present invention are suitable for collecting saliva specimens.

For a second experiment, 35 mL of saliva specimen (Pooled normal saliva from human donors, Catalog no. 991-05-P; Lee Biosolutions). The saliva was spiked with a known quantity of heat-inactivated virus (325 μL of SARS-Related Coronavirus 2, Isolate USA-WA1/2020, Catalog No. NR-52286, BEI Resources) to obtain a spiked sample at approximately 90 copies/mL. The spiked sample was then mixed with equal quantity of Composition C1 (Table 1). Six aliquots of 1.5 mL each were stored at 30° C. and tested for SARS-COV-2 after 1, 2, 3, 5, 7 and 10 of days of storage. Eight aliquots of 1.5 mL each were stored at 4° C. and tested for SARS-COV-2 after 1, 2, 3, 5, 7, 10, 13 and 15 days of storage. All aliquots were tested in 5 replicates by processing in the Panther System as previously explained. One aliquot of 1.5 mL was tested at Day 0 as baseline. The results are shown in Table 17. The samples showing positive results for SARS-COV-2 were considered reactive, while a valid sample implies that the sample was suitably processed by the Panther System.

TABLE 17
Preservation of saliva samples

| Condition | | | # | # | | | % |
|---|---|---|---|---|---|---|---|
| Temperature | | Day | Samples | Replicates | Valid | Reactive | Reactive |
| 30° | C. | 1 | 1 | 5 | 5 | 5 | 100 |
| | | 2 | 1 | 5 | 5 | 5 | 100 |
| | | 3 | 1 | 5 | 5 | 5 | 100 |
| | | 5 | 1 | 5 | 5 | 5 | 100 |
| | | 7 | 1 | 5 | 5 | 5 | 100 |
| | | 10 | 1 | 5 | 5 | 5 | 100 |
| 4° | C. | 1 | 1 | 5 | 5 | 5 | 100 |

| | | | | | | |
|---|---|---|---|---|---|---|
| | 2 | 1 | 5 | 5 | 5 | 100 |
| | 3 | 1 | 5 | 5 | 5 | 100 |
| | 5 | 1 | 5 | 5 | 5 | 100 |
| | 7 | 1 | 5 | 5 | 5 | 100 |
| | 10 | 1 | 5 | 5 | 5 | 100 |
| | 13 | 1 | 5 | 5 | 5 | 100 |
| | 15 | 1 | 5 | 5 | 5 | 100 |
| Baseline | 0 | 1 | 5 | 5 | 5 | 100 |

This experiment shows that the compositions of the present invention preserve the RNA content of saliva specimens for at least 10 days at 30° C. and for at least 15 days at 4° C.

1. An aqueous composition for storing a biological sample for subsequent nucleic acid testing, the composition comprising
a first component comprising:
i) an anionic detergent in an amount from about 1% to 20% (w/v),
ii) a Group I metal hydroxide in an amount from about 1% to 5% (w/v),
iii) a chelating agent in an amount from about 0.5% to 5% (w/v), and
iv) a first buffer, wherein
the composition further comprises a diluting component selected from the group consisting of water, a second buffer, a Transport Medium, Sodium Chloride (aq), and combinations thereof,
wherein the first component is diluted up to a maximum of about 50% (v/v) with the diluting component.

2. The aqueous composition of claim 1, wherein the component is Sodium Chloride (aq).

3. The aqueous composition of anyone of claim 1 or 2, wherein the anionic detergent comprises a C5-C20 alkyl sulfate anion, a C5-C20 alkenyl sulfate anion, a C5-C20 alkynyl sulfate anion, or any combination thereof.

4. The aqueous composition of claim 3, wherein the detergent comprises a lauryl sulfate anion.

5. The aqueous composition of anyone of claims 1 to 4, wherein the first buffer is selected from Tris, MES, Bis-Tris, HEPES, MOPS, citrate, sodium bicarbonate, sodium phosphate, and combinations thereof.

6. The aqueous composition of claim 5, wherein the first buffer comprises HEPES.

7. The aqueous composition of anyone of claims 1 to 6, wherein the first buffer is present in an amount from about 5% to 30% (w/v).

8. The aqueous composition of one of claims 1 to 6, wherein the first buffer is present in a sufficient amount to maintain a pH between 5 and 10.

9. The aqueous composition of anyone of claims 1 to 8, wherein the Group I metal hydroxide is selected from lithium hydroxide, sodium hydroxide, potassium hydroxide, and combinations thereof.

10. The aqueous composition of claim 9, wherein the Group I metal hydroxide comprises lithium hydroxide.

11. The aqueous composition of anyone of claims 1 to 10, wherein the chelating agent is selected from EGTA, HEDTA, DTPA, NTA, EDTA, succinic acid, citrate anhydrous, sodium citrate, calcium citrate, ammonium citrate, ammonium bicitrate, citric acid, diammonium citrate, potassium citrate, magnesium citrate, ferric ammonium citrate, lithium citrate, and combinations thereof.

12. The aqueous composition of claim 11, wherein the chelating agent is selected from succinic acid, EGTA, EDTA, and combinations thereof.

13. The aqueous composition of anyone of claims 1 to 12, wherein the first buffer is present in an amount from about 10% to 25% (w/v), the anionic detergent is present in an amount from about 2% to 15% (w/v), the chelating is present in an amount from about 1% to 4% (w/v), and the Group I metal hydroxide is present in an amount from about 1% to 4% (w/v).

14. The aqueous composition of anyone of claims 1 to 13, wherein the pH of the aqueous composition is between 5 and 10.

15. The aqueous composition of claim 14, wherein the pH of the aqueous composition is between 6 and 9.

16. The aqueous composition of anyone of claims 1 to 15, further comprising an antifoaming agent in amount from about 50 μl/L to 750 μl/L.

17. The aqueous composition of claim 16, wherein the antifoaming agent comprises a silicone polymer, a polysorbate, an organic polyether dispersion or any combination thereof.

18. The aqueous composition of anyone of claims 1 to 17, further comprising a population of polynucleotides that comprises RNA, DNA, or any combination thereof.

19. The aqueous composition of claim 18, wherein the RNA comprises an in vitro synthetized transcript.

20. An aqueous composition for storing a biological sample for subsequent nucleic acid testing, the composition comprising:
i) an anionic detergent in an amount from about 1% to 20% (w/v),
ii) a Group I metal hydroxide in an amount from about 1% to 5% (w/v),
iii) a chelating agent in an amount from about 0.5% to 5% (w/v), and
iv) a first buffer,
wherein the composition comprises an antifoaming agent in amount from about 50 μl/L to 750 μl/L.

21. The aqueous composition of claim 20, wherein the antifoaming agent comprises a silicone polymer, a polysorbate, an organic polyether dispersion, or any combination thereof.

22. The aqueous composition of anyone of claim 20 or 21, wherein the anionic detergent comprises a C5-C20 alkyl sulfate anion, a C5-C20 alkenyl sulfate anion, a C5-C20 alkynyl sulfate anion, or any combination thereof.

23. The aqueous composition of claim 22, wherein the detergent comprises a lauryl sulfate anion.

24. The aqueous composition of anyone of claims 20 to 23, wherein the first buffer is selected from Tris, MES, Bis-Tris, HEPES, MOPS, citrate, sodium bicarbonate, sodium phosphate, and combinations thereof.

25. The aqueous composition of claim 24, wherein the first buffer comprises HEPES.

26. The aqueous composition of anyone of claims 20 to 25, wherein the first buffer is present in an amount from about 5% to 30% (w/v).

27. The aqueous composition of anyone of claims 20 to 25, wherein the first buffer is present in a sufficient amount to maintain a pH between 5 and 10.

28. The aqueous composition of anyone of claims 20 to 27, wherein the Group I metal hydroxide is selected from lithium hydroxide, sodium hydroxide, potassium hydroxide, and combinations thereof.

29. The aqueous composition of claim 28, wherein the Group I metal hydroxide comprises lithium hydroxide.

30. The aqueous composition of anyone of claims 20 to 29, wherein the chelating agent is selected from EGTA, HEDTA, DTPA, NTA, EDTA, succinic acid, citrate anhydrous, sodium citrate, calcium citrate, ammonium citrate, ammonium bicitrate, citric acid, diammonium citrate, potassium citrate, magnesium citrate, ferric ammonium citrate, lithium citrate, and combinations thereof.

31. The aqueous composition of claim 30, wherein the chelating agent is selected from succinic acid, EGTA, EDTA, and combinations thereof.

32. The aqueous composition of anyone of claims 20 to 31, wherein the first buffer is present in an amount from about 10% to 25% (w/v), the anionic detergent is present in an amount from about 2% to 15% (w/v), the chelating is present in an amount from about 1% to 4% (w/v), and the Group I metal hydroxide is present in an amount from about 1% to 4% (w/v).

33. The aqueous composition of anyone of claims 20 to 32, wherein the pH of the aqueous composition is between 5 and 10.

34. The aqueous composition of claim 33, wherein the pH of the aqueous composition is between 6 and 9.

35. The aqueous composition of anyone of claims 20 to 34, wherein the composition is diluted up to a maximum of about 50% (v/v) with a component selected from the group consisting of water, a second buffer, a Transport Medium, Sodium Chloride (aq), and combinations thereof.

36. The aqueous composition of claim 35, wherein the component is Sodium Chloride (aq).

37. The aqueous composition of anyone of claims 20 to 36, further comprising a population of polynucleotides that comprises RNA, DNA, or any combination thereof.

38. The aqueous composition of claim 37, wherein the RNA comprises an in vitro synthetized transcript.

39. A method for obtaining a population of polynucleotides from a sample suspected of containing nucleic acids, said method comprising contacting the sample with the aqueous composition according to anyone of claims 1 to 38, at a temperature ranging from 2° C. to 40° C.

40. The method of claim 39, wherein the sample is a biological sample or an environmental sample.

41. The method of anyone of claim 39 or 40, wherein the nucleic acids are RNA and/or DNA.

42. The method of claim 41, wherein the RNA comprises an in vitro synthetized transcript.

43. The method of anyone of claims 39 to 42, wherein the nucleic acids are from at least one pathogen.

44. The method of claim 43, wherein the at least one pathogen is a fungi, a bacteria, a parasite or a virus.

45. The method of claim 44, wherein the at least one pathogen is a fungi.

46. The method of claim 44, wherein the at least one pathogen is a bacteria.

47. The method of claim 44, wherein the at least one pathogen is a parasite.

48. The method of claim 44, wherein the at least one pathogen is a virus.

49. The method of claim 48, wherein the virus is SARS-COV-2.

50. The method of anyone of claims 39 to 49, further comprising an amplification reaction to amplify at least a target nucleic acid and the detection of at least one target of the resulting amplification product.

51. A method for preserving the integrity of a population of polynucleotides in a sample, comprising contacting the sample with the aqueous composition according to anyone of claims 1 to 38, at a temperature ranging from 4° C. to 40° C.

52. The method of claim 51, wherein the sample, after being contacted with the aqueous composition, is stored at a temperature ranging from −25° C. to 40° C. to for at least 1 hour up to 6 months, up to 9 months, up to 12 months, or up to 26 months.

53. The method of claim 51, wherein the sample, after being contacted with the aqueous composition, is stored at −20° C., 4° C., RT or 30° C.

54. The method of anyone of claims 51 to 53, wherein the sample is a biological sample or an environmental sample.

55. The method of anyone of claims 51 to 54, wherein the population of polynucleotides is a population of RNA and/or DNA.

56. The method of claim 55, wherein the RNA comprises an in vitro synthetized transcript.

57. The method of anyone of claims 51 to 56, wherein the population of polynucleotides are from at least one pathogen.

58. The method of claim 57, wherein the at least one pathogen is a fungi, a bacteria, a parasite or a virus.

59. The method of claim 58, wherein the at least one pathogen is a fungi.

60. The method of claim 58, wherein the at least one pathogen is a bacteria.

61. The method of claim 58, wherein the at least one pathogen is a parasite.

62. The method of claim 58, wherein the at least one pathogen is a virus.

63. The method of claim 62, wherein the virus is SARS-COV-2.

64. A method for inactivating a sample comprising at least one pathogen, the method comprising contacting the sample with the aqueous composition according to anyone of claims 1 to 38, wherein the aqueous composition represents at least 30% (v/v) of the resulting mixture.

65. The method of claim 64, wherein the sample is contacted with the aqueous composition at 4° C., RT or 30° C.

66. The method of anyone of claim 64 or 65, wherein the sample is a biological sample or an environmental sample.

67. The method of anyone of claims 64 to 66, wherein the at least one pathogen is a fungi, a bacteria, a parasite or a virus.

68. The method of claim 67, wherein the at least one pathogen is a fungi.

69. The method of claim 67, wherein the at least one pathogen is a bacteria.

70. The method of claim 67, wherein the at least one pathogen is a parasite.

71. The method of claim 67, wherein the at least one pathogen is a virus.

72. The method of claim 71, wherein the virus is SARS-COV-2.

73. A method for collecting a sample comprising contacting the sample with the aqueous composition according to anyone of claims 1 to 38, wherein the aqueous composition represents at least 30% (v/v) of the resulting mixture.

74. The method of claim 73, where the sample is a biological sample or an environmental sample.

75. The method of anyone of claim 73 or 74, wherein the sample is directly introduced into a collection device or a collection vessel comprising the aqueous composition according to any one of claims 1 to 38.

76. The method of anyone of claim 73 or 74, wherein the sample is collected using a swab and introducing it into a collection device or a collection vessel comprising the aqueous composition according to anyone of claims 1 to 38.

77. The method of claim 76, wherein the swab is discarded after rubbing it against the collection device or the collection vessel.

78. The method of anyone of claims 73 to 77, wherein the sample comprises at least one pathogen.

79. The method of claim 78, wherein the at least one pathogen is a fungi, a bacteria, a parasite or a virus.

80. The method of claim 79, wherein the at least one pathogen is a fungi.

81. The method of claim 79, wherein the at least one pathogen is a bacteria.

82. The method of claim 79, wherein the at least one pathogen is a parasite.

83. The method of claim 79, wherein the at least one pathogen is a virus.

84. The method of claim 83, wherein the virus is SARS-COV-2.

85. A collection device or vessel comprising the aqueous composition according to anyone of claims 1 to 38.

86. A sample collection kit comprising a collection device or a collection vessel and the aqueous composition according to anyone of claims 1 to 38.

87. The sample collection kit of claim 86, further comprising a swab, a curette, or a culture loop.

88. Use of the aqueous composition of anyone of claims 1 to 38 to collect a sample suspected of containing a target nucleic acid.

89. The use of claim 88, wherein the sample is a biological sample or an environmental sample.

90. The use of anyone of claim 88 or 89, wherein the sample comprises at least one pathogen.

91. The use of claim 90, wherein the at least one pathogen is a fungi, a bacteria, a parasite or a virus.

92. The use of claim 91, wherein the at least one pathogen is a fungi.

93. The use of claim 91, wherein the at least one pathogen is a bacteria.

94. The use of claim 91, wherein the at least one pathogen is a parasite.

95. The use of claim 91, wherein the at least one pathogen is a virus.

96. The use of claim 95, wherein the virus is SARS-COV-2.
